(19) United States
(12) Patent Application Publication
(10) Pub. No.: US 2010/0291160 A1
(43) Pub. Date: Nov. 18, 2010

(54) PHARMACEUTICAL SYSTEM FOR TRANS-MEMBRANE DELIVERY

(76) Inventors: David R. Carver, Colorado Springs, CO (US); Troy G. Fohrman, Palm City, FL (US); Sean William Reynolds, Colorado Springs, CO (US)

Correspondence Address: WINSTON & STRAWN LLP;PATENT DEPARTMENT, 1700 K STREET, N.W., WASHINGTON, DC, 20006, US

(21) Appl. No.: 12/779,017

(22) Filed: May 12, 2010

Related U.S. Application Data
(60) Provisional application No. 61/177,753, filed on May 13, 2009.
(60) Provisional application No. 61/243,338, filed on Sep. 17, 2009.

(51) Int. Cl. A61K 9/00; A61K 31/519; A61K 38/16; A61K 31/46; A61K 38/19; A61K 31/727; A61K 38/29; A61K 39/395; A61K 31/137; A61P 29/00; A61P 9/06; A61P 7/02; A61P 3/02; A61P 19/10; A61P 21/00; A61P 5/00; A61P 31/04; A61P 31/10; A61P 9/12; A61P 3/10; A61P 3/04; A61P 1/04; A61P 1/08; A61P 25/16; A61P 25/22

(52) U.S. Cl. 424/400; 514/252.16; 514/21.1; 514/304; 424/85.1; 514/56; 514/11.8; 424/142.1; 514/653

(57) ABSTRACT

Non-invasive drug delivery systems useful for the absorption of therapeutically active agents through the epithelial membrane are described. The non-invasive drug delivery system delivers a therapeutic active agent with an ionizable, or ionized, metal, transition metal or metal-containing vehicle. The non-invasive drug delivery system may also have a pH adjustable vehicle which facilitates the absorption of the therapeutic agents by altering the pH of the non-invasive drug delivery system at the site of administration. Also disclosed is a method for the pH “sweeping” of the administered therapeutic active agent to provide a consistent and reproducible absorption of the active agent. Certain formulations utilize low doses of active agents without altering the active agents from their current or previous form.

### RELATED PATENT APPLICATIONS

This application claims the benefit of U.S. Provisional Patent Application No. 61/177,753 filed May 13, 2009, and U.S. Provisional Patent Application No. 61/243,338 filed Sep. 17, 2009, which are both hereby incorporated herein by reference in their entireties.

### FIELD OF THE INVENTION

The present invention relates to a non-invasive system and methods for absorption of therapeutic active agents through the epithelial membranes of a patient. The present system also relates to methods for the production of metal-complexed vehicles for the non-invasive transport of therapeutic active agents, as well as novel systems produced thereby, and methods of treatment utilizing the systems produced herein.

### BACKGROUND OF THE INVENTION

The sudden onset of allergic reactions initiates a cascade of physiologic changes which may culminate in anaphylactic shock. Loss of adequate insulin levels in a diabetic can result in a diabetic coma. The conventional treatment is immediate treatment with the proper therapeutic agent. It is understood, however, that rapid administration of effective amounts of therapeutic agents can be as important for controlling or preventing a disease or condition as the therapeutic agent itself.

In the examples of anaphylactic shock and diabetes above, the treatment must be given immediately and by injection. Despite the obvious aversion of most people, particularly children, to a self-administered injection, there are other potential difficulties, such as breakage of the syringe or injectable device, expiration of the drug contents, and most commonly, unavailability because the injectable delivery system is inconvenient to carry. Moreover, in situations in which the administration of an oral medication is necessary during an acute event, such as a myocardial infarction or a seizure, the patient may not physically be able to ingest the medication, or have sufficient time between ingestion of the therapeutic agent and absorption into the blood stream, for the therapeutic agent to provide a therapeutic benefit, such as reduction or relief of symptoms. There is a need in the art for a more convenient, less invasive, preferably non-invasive, treatment system that provides rapid or more immediate release of the therapeutic agent.

Immediate, non-invasive rapid release systems have not been successfully developed. A major obstacle to the development of such a non-invasive release delivery systems has been that existing technologies have often resulted in the conjugation of the delivery agent to the therapeutic active agent, generally forming an unintended new and distinct agent that often has new properties and safety profile different from the original therapeutic active agent, and are effective treatments for the original indication.

Another major obstacle to the development of a non-invasive delivery system is the uncontrolled and inconsistent delivery of pharmacologically active agents. Non-optimal patient outcomes are often due to low or highly-variable levels of bioavailability of the active agent. Many oral delivery systems have shown poor absorption in the gastrointestinal (GI) tract, and therefore require increased dosing. As doses increased, an increased level of associated toxicities is also typically observed.

Yet another problem in the development of non-invasive delivery systems is that these systems have not been shown to be successful at transporting molecules, particularly of a very small or very large size. In fact, the conventional art has shown that there is difficulty transporting active agents from about 1 to about 500 KiloDaltons (kD) through membranes.

Another drawback to the development of non-invasive delivery systems is their reliance on the degradation of mammalian membrane(s) in order to administer active agents. The degradation of the membranes can cause severe irritation, sores, and discomfort to the patient. The placement of the delivery device (patch, gel, etc.) must be relocated for subsequent administrations in order to allow previous absorption sites to heal.

Additionally, prior art non-invasive oral delivery formulations have generally been shown to be effective only if properly administered by the patient. However, data suggests that sublingual-type formulations are frequently improperly administered. For example, the patient may fail to understand that sublingual involves dissolving medicine under the tongue, and that absorption in this manner is more effective for certain dosages than simply chewing and swallowing.

Some non-invasive formulations for promotion of DNA into the cell of a mammal, such as those described in U.S. Pat. No. 6,624,149, do not embody the use of transition metal enhancers outside of a cationic lipid. Other prior art formulations, such as those described in U.S. Patent Application Publication No. 2008/0242595, have the active agent, insulin, covalently bonded to vitamin B12. This conjugation of insulin to vitamin B12 resulted in a new therapeutic active agent, distinct from either vitamin B12 or insulin.

As such, a need exists for improved non-invasive formulations in the field of immediate-release medicaments. More specifically, there is a need for a delivery system that may deliver an active agent in an effective amount without degradation or other loss of therapeutic activity.

### SUMMARY OF THE INVENTION

In accordance with the principles of the present invention, methods, compositions, and systems for non-invasive delivery systems are provided. In one embodiment, a non-invasive delivery system (e.g., a tablet, patch or lotion) for delivering an active agent by absorption across a cellular membrane is provided. The non-invasive delivery system comprises: (a) an effective amount of an active agent; and (b) at least one vehicle for facilitating absorption of the active ingredient across a cellular membrane, preferably mucosal or epithelial membranes, said vehicle being selected from the following: (i) metal-complexed vehicles that reversibly complex with the active agent through ionic interactions; or (ii) pH-adjusting vehicles that adjust the pH of the non-invasive delivery system from a first pH to a second pH.

Preferred embodiments of the delivery system include at least the effective amount of active agent ionically interacting (or ionically bound) to a metal-complexed vehicle in conjunction with a pH-adjusting vehicle. Embodiments may further include additional agents to facilitate transport of the active agent through the membrane. Preferably, the metal-compound vehicle includes a metallic component, most preferably a vitamin B12 metallic complex or chlorophyllin. In more preferred embodiments the vitamin B12 is associated with cobalt, often referred to as cobalamin.

In other embodiments, the non-invasive delivery system can include at least one metal-complexed vehicle that includes at least one of the following: an ionizable transition metal, an ionized transition metal, an ionizable metal-containing compound or complex, or an ionized metal-containing compound or complex. In alternate embodiments, the non-invasive delivery system includes at least a salt that is ionizable or ionized, or the system includes at least an amino acid, protein, peptide, sugar, a detergent, or a combination thereof. Preferably, the amino acid is arginine. In certain embodiments, the non-invasive delivery system includes at least a pH buffer. In preferred embodiments, the pH-adjusting vehicle of the non-invasive delivery system includes an acidic component and a basic component.

Certain additional embodiments are directed to methods of treating a disease or condition in a mammal, preferably a human subject, including the steps of administering a non-invasive delivery system that comprises an effective amount of an active agent and at least one metal-complexed vehicle or at least one pH-adjusting vehicle, preferably both a metal-complexed vehicle and a pH-adjusting vehicle, wherein the at least one metal-complexed vehicle or the at least one pH-adjusting vehicle reversibly complexes with the active agent through ionic interactions and preferably facilitates absorption of the active agent across a membrane.

In particular embodiments, the method includes determining a desired pH level for transport and/or pH level associated with the active agent. The pH-adjusting vehicle and/or other components of the delivery system affecting pH may be adjusted to provide a pH sweep targeted to the desired, or necessary, pH optimized for the active agent. In some embodiments, the at least one metal-complexed vehicle or at least one pH-adjusting vehicle is administered dermally or through a mucosal membrane, such as sublingually. In alternate embodiments, the pH-adjusting vehicle is provided in an amount sufficient to alter the pH of the non-invasive delivery system from a first pH value to a second pH value in order to facilitate absorption of the active agent across the membrane. In another embodiment, the metal-complexed vehicle includes at least one of the following: an ionizable or ionized transition metal or metal-containing compound that preferably changes oxidation states across the membrane in order to facilitate absorption of the active agent across the membrane.

In an additional embodiment, the non-invasive delivery system may include at least an ionized salt, or a readily ionizable salt, wherein the ionized salt, or readily ionizable salt provides ionic strength to the non-invasive system equal to the ionic strength of the epithelial membrane in order to facilitate absorption of the active agent across a membrane.

The system and methods disclosed herein, demonstrate a variety of unique and novel adjuvants that, surprisingly in combination, can be added to active agents in the delivery system to affect an enhanced absorption of the active agent across a membrane. The active agent is preferably ionically bound to the vehicles of the delivery system. More preferably, the interaction between the active agent and the delivery system is reversible. Certain embodiments, such as the Wet Induction Sublingual Entry System™ (WISE) (Protein Delivery Solutions, LLC, Lantana, Fla.), may include systems and methods to enhance and/or control absorption of unaltered active agents through the use of various vehicles that may adjust preferably the pH and ionic strength, as well as may even more preferably, other factors that allow the rapid absorption of the active agents. The metal-complexed vehicles can include a pH adjusting agent that allows the system to provide a “pH sweep” of the system at the site of delivery in order to maximize the absorption of the active agent.

While oral dosages forms may take any shape that allows for oral use by the patient, it is preferable to provide a boomerang-shaped or horseshoe-shaped sublingual oral form, which enhances the use by the patient's visual recognition cues for proper placement of the dosage form in the oral cavity. Proper placement of the dosage form as a sublingual medicament would increase dissolution and absorption of the medicine.

### BRIEF DESCRIPTION OF THE DRAWINGS

Further features of the invention, its nature and various advantages will be more apparent from the following detailed description, taken in conjunction with the accompanying drawings in which like reference characters refer to like parts throughout and in which:

FIG. 1 is an exemplary representation of the delivery system, in accordance with an embodiment of the invention;

FIG. 2 is an alternate view of an exemplary representation of the delivery system, in accordance with one embodiment of the invention;

FIG. 3 is a schematic diagram of an exemplary formulation of the delivery system, in accordance with an embodiment of the invention;

FIG. 4 is graphical depiction of an exemplary formulation of the delivery system, in accordance with one embodiment of the invention;

FIG. 5 is a cross-section of an exemplary multilayer delivery system, in accordance with one embodiment of the invention; and

FIG. 6 is a cross-section of another exemplary multilayer delivery system, in accordance with one embodiment of the invention.

### DETAILED DESCRIPTION OF PREFERRED EMBODIMENTS

The present invention discloses non-invasive delivery systems and methods that provide an effective administration to mammals, preferably humans, of one or more active pharmacological ingredients (i.e., “active agent”) through cellular membranes. In preferred embodiments, the delivery system comprises a mixture of vehicles, which form ionic interactions with the active agent. Surprisingly, the combination of vehicles and other ingredients enhance absorption of the active agent, preferably in a non-degraded form, across a cellular or biological membrane. Through the use of various ingredients (interchangeably referred to herein as “adjuvants”) the system and methods may adjust the pH, ionic strength, and other factors affecting the permeability of the membrane that would allow or enhance the rapid absorption of the active agents, preferably in an unaltered, pharmaceutically effective and therapeutically effective state. Moreover, embodiments of the present invention provide novel solutions to both invasive protein delivery and improved bioavailability for therapeutic active agents. The high efficiency of this delivery system combined with the nearly immediate bioavailability of the therapeutic active agent(s), allow for decreased dosing and thus a decreased level of associated toxicities.

Certain embodiments of the present system relate to methods for the administration and processes of making delivery systems having one or more vehicles, preferably a metal-complexed vehicle, a pH-adjusting vehicle, or a combination of thereof, for the non-invasive intra-membrane transport of unaltered therapeutic active agents through cellular membranes. In preferred embodiments, the system and methods include metal-complexed vehicles in combination with a pH-adjusting vehicle, which provides a “pH sweep” of the system at the site of delivery in order to maximize the absorption of the active agent.

The present system provides numerous advantages over conventional treatments. For example, preferred embodiments provide a more easily administered delivery system which would allow a patient in need thereof to administer treatment, not as an injection, but as an oral self-administered, preferably sublingual dosage, such as a tablet, film or liquid. The oral dosage would increase access to, and ease of use of the medication, resulting in greater usage and increased patient acceptance, particularly in pediatric populations. An additional benefit is an increase in stability of the formulations, particularly formulations having biologically and chemically unstable molecules, such as proteins. The delivery systems of the present invention would also be adaptable for use to a wide range of active ingredients, as the delivery system may handle active ingredients ranging up to 1000 kD. Preferred embodiments are capable of transporting molecules less than about 0.01 kD and greater than about 200 kD across membranes.

For the purposes herein, the term “non-invasive delivery system” or “non-invasive system” includes one or more formulations facilitating or providing the absorption of an active agent, preferably in a physiologically unaltered state, across a cellular membrane. The non-invasive system may provide for the transportation of unaltered active agents across mammalian membranes by programming pores, channels and/or intercellular channels within the mammalian membranes in order to facilitate absorption. Preferably, transport of the active agent through the membrane is passive, such as by diffusion, facilitated diffusion and/or osmosis. In certain embodiments, transportation is active, including primary and/or secondary active transport.

As used herein, “pore programming” refers to the ability to modulate the pores of a membrane in order to facilitate absorption. Preferably pore programming can be individualized to each active agent as required. Moreover, it is preferred that while using pore programming that the membrane's integrity remains substantially intact.

As used herein, the term “membrane” refers to a selective cellular barrier that is selectively permeable and controls the movement of substances into and out of cells. Membranes generally comprise proteins and lipids. Additionally, membranes include a cell potential. Membranes encompass all cellular membranes, preferably animal, more preferably mammalian, and most preferably human. The membrane can be without limitation, both connective and epithelial membranes. An example of a connective membrane includes a synovial membrane. Examples of epithelial membranes include the skin, mucosal membranes and serous membranes. The membranes can be dry or wet membranes. Examples of additional mammalian membranes, for both humans and other mammals, include mesenteric, dermal, epidermal, blood-brain barrier, intervaginal, rectal, ocular, internasal and tempanic membranes.

Embodiments of the system may provide delivery of physiological active agents to the blood stream without passing through the GI tract. Preferred embodiments allow the active ingredient to enter specific organs, such as the skin for dermal applications and the blood stream for sublingual applications. In certain embodiments, the delivery system provides targeted delivery of the active agent. For example, the blood supply directly would receive the active agent without having the active agent pass through to the liver directly via the GI tract. Elimination of the “first-pass” detoxification allows for a decreased dose of the therapeutic active agent, decreased toxicity from metabolic by-products of liver detoxification, and enhanced speed of delivery of the therapeutic active agent.

Embodiments of the present invention are intended for administration to animals in need, particularly mammals, and more particularly human subjects. Accordingly, for purposes herein, the term “patient” or “subject” refers to an individual who is in need of an active agent for therapeutic, prophylactic, or preventative reasons, preferably as prescribed by a medical or clinical professional. A patient can refer to a mammal, including, but not limited to humans, monkeys, rats, cows, sheep, dogs, cats, goats, etc. A patient may refer to an adult or a child.

For the purposes herein, the term “active agent” refers to any molecule administered for a therapeutic benefit. An active agent is preferably a pharmacologically active agent. An active agent can be physiologically active. An active agent may refer to, without limitation, pharmaceuticals, including large-molecule pharmaceuticals, small-molecule pharmaceuticals, biopharmaceuticals (also referred to as “biologics”), large-biopharmaceuticals, small-molecule biopharmaceuticals, nutraceuticals, genetic material (including DNA and RNA) preferably isolated or purified, recombinant nucleic acid vectors (e.g., as plasmids, cosmids, etc.), vaccines, proteins, peptides, hormones, organic or inorganic molecules, nanoparticles (e.g., nanocarbons, nanodiamonds, silicons, sulfates/sulfites technologies) or any combination thereof. Examples of proteins/peptides may include antibodies (MAbs), glycoslated molecules, fusion proteins, protein fragments, sterols and bioidentical compounds as well as a wide variety of combinations of amino acids. Additional examples of suitable active agents are provided herein.

One or more active agents may be used. A combination of active agents may be provided concurrently or serially, preferably in any order. The size of the active agent may range between from about 0 kD to about 500 kD or more. One skilled in the art may prefer to use such a formulation for the non-invasive delivery of active agents that range from about 0 to about 20 kD, while other skilled artisans prefer to use the formulation with active agents from about 2 to about 200 kD, or from about 200 kD to about 500 kD or more. In some embodiments, the active ingredient may range up to about 1000 kD.

For the purposes herein, an “unaltered” therapeutic active agent refers to an unchanged or unaltered active agent when transported. An unchanged therapeutic active agent is an active agent that undergoes no molecular or irreversible changes. Preferably, the active agent does not undergo any irreversible changes in chemical structure, properties, or activity. Even more preferably, the active agents in the present disclosure are transported across the membranes in their original state. It is yet more preferred that the system maintains, or at least does not substantially decrease, the pharmaceutical and/or therapeutic effect of the active embodiments, the delivery system provides the patient in need with out an effective amount of active agent. For example, an unaltered active agent is not glycosolated solely for the purposes of absorption (wherein the therapeutic agent may not glycosolated). As another example, the unaltered active agent does not become conjugated to another molecule for the purposes of absorption (wherein the therapeutic active agent is not normally conjugated to another molecule). Still, as another example, an unaltered active agent is not cleaved into a smaller portion or fragment in order for successful absorption (absorption occurs with the active agent wholly intact).

In preferred embodiments the vehicles of the system and the active agent are not covalently bonded. In the most preferred embodiments, the active agent and non-invasive system are bound together through ionic interactions. Interactions of the active agent with the non-invasive delivery system do not require, and preferably avoid conjugation, or the formation of new moieties, that may reduce the therapeutic effectiveness of the active agents. It is preferable that the system does not change the stoichiometry or physiologic functions of the treating agent, and thus, the physiologic therapeutic effect of the active agent is preserved.

The term “an effective amount,” as used herein, is encompassed by the above-described dosage amounts and dose frequency schedule, particularly when coupled with prevention, treatment, or management of one or more conditions requiring therapeutic treatment. Effective amounts may vary by subject, disease and active agent, but are generally known in the art or are determinable or optimizable by routine testing.

For the purposes herein, “prophylaxis” may refer to prevention of the symptoms of a disease, a delay in onset of the symptoms of a disease, or a lessening in the severity of subsequently developed disease symptoms. The terms “prevent,” “preventing,” and “prevention” refer herein to the inhibition of the development or onset of a disorder or the prevention of the recurrence, onset, or development of on or more symptoms of a disorder in a subject resulting from the administration of a therapy (e.g., a prophylactic or therapeutic agent), or the administration of a combination of therapies (e.g., a combination of prophylactic or therapeutic agents). For the purposes herein, “therapy” or “treatment” or “treat” can mean a complete abolishment of the symptoms of a disease or a decrease in the severity of the symptoms of the disease.

For the purposes herein, the term “substantially,” is intended to include variations from the absolute condition, e.g., about 90 percent, preferably about 95 percent, more preferably about 99 percent of the absolute condition. In preferred embodiments, the term “substantially” refers to 99.9 percent or even 99.99 percent of the absolute.

For the purposes herein, the term “pH sweep” refers to the system's capability to alter the immediate environment surrounding the formulation by progressing from an acidic pH to a basic pH, or from a basic pH to an acidic pH. The pH sweep creates a tight pH control in the delivery area related to the pore(s), channel(s), and/or intercellular channel(s) on the mammalian membrane(s). As a result, this pH sweep maintains an optimal environment for the transport of the active agent(s), while preferably avoiding the degradation of the agent(s) in an uncomplexed state under normal biologic function. Preferably, the pH sweep is effective at, or substantially at, the transport site of the membrane.

For the purposes herein, the term “immediate environment” refers to the area at, or substantially near, where the formulation is directly administered. Preferably, the immediate environment is directly at the site of administration when the formulation is administered. Preferably, the immediate environment includes both the formulation and the site of administration when the formulation is administered and may be in contact with the patient. The immediate environment may allow for the formulation to interact directly with the membrane to allow the system to facilitate absorption. For example, the immediate environment may allow a dehydrated formulation applied to a site of administration to hydrate using moisture wicked from the site of administration. In another example, the immediate environment may allow a pH-adjusting agent to facilitate absorption by promoting the interaction of the acidic component with the basic component at the site of absorption. In some embodiments, the immediate environment is within about 0-5 cm or less of the administered system formulation.

Natural mammalian membranes typically have a pH range from about 2 to about 10. In preferred embodiments, the pH sweep can occur such that the range that is “swept” ranges from 1, 2, 3 or more pH values. More preferably, the pH sweep can range from a pH of about 2 to a pH of about 10. In other embodiments, the pH sweep can range from a pH of about 10 to a pH of about 2. The pH sweep can occur such that the range that is “swept” ranges over whole or fractions of a pH value. For example, a pH sweep may raise the pH from about 6 to about 7, about 7 to about 8, or from about 6.5 to about 8.5. Alternative examples can include a pH sweep that lowers the pH from about 8 to about 7, about 7.5 to about 6 or from about 8.5 to about 6.5. The pH sweep may preferably occur as the formulation dissolves at the site of absorption or over a relatively rapid time. The pH sweep may also occur from about 1 to about 10 minutes or less after administration. In alternative examples, the pH sweep can occur from about 1 to about 360 seconds, preferably from about 1 to about 180 seconds, or preferably about 1 to about 120 seconds, even more preferably about 1 to about 90 seconds, yet more preferably from about 1 to 60 seconds, and most preferably from about 1 to about 30 seconds after administration. In most preferred embodiments, the pH sweep may occur sufficient to allow complete or substantially complete absorption of the active agent.

For the purposes herein, the term “metal-complexed vehicles” refers to an ionizable or ionized transition metal or metal-containing compound complexed to an active ingredient, for example a transport molecule, which may be comprised of an amino acid, peptide, protein, sugar, and/or detergent that is capable of facilitating the transport of an active agent across a membrane. Metal-complexes may be characterized as compounds that are metal-containing cofactors that interact with macromolecules to promote biological processes. Metal-complexes used in embodiments of the present invention may include porphyrins, metalloproteins, and metalloenzymes. A metal-complexed vehicle may include vitamin B12 and its various forms such as cyanocobalamin, adenosylcobalamin, hydroxocobalamin, and preferably cobalamin. Another metal-complexed vehicle may include chlorophyll or chlorophyll-complexes which may include copper.

For the purposes herein, the term “complex” of “complexed” refers to two or more components of the delivery system that held together via ionic, or hydrogen interactions, which allows the delivery system to deliver unaltered active agents across a membrane.

For the purposes herein, the term “pH adjusting vehicle” refers to a complex comprising at least an acidic component or a basic component. Preferably, the pH adjusting vehicle can comprise an acidic component and/or a basic component. The acidic component may be separated from the basic component prior to administration. The acidic component may be in direct contact with the basic component at the time of administration. The pH adjusting vehicle can facilitate the absorption of active agents across a membrane by facilitating a pH sweep in order to optimize absorption conditions at the site of administration.

Embodiments of the non-invasive delivery system include various combinations of the following six ingredient categories:
Ingredient A: an ionizable or ionized transition metal or metal-containing compound;
Ingredient B: a salt that is ionized or readily ionizable;
Ingredient C: an amino acid, peptide, protein, sugar, and/or detergent that will readily, but reversibly, bind to ingredients A and B;
Ingredient D: a buffered solution, film or powder to optimize the pH of the mixture;
Ingredient E: the therapeutic active agent(s); and
Ingredient F: a solid or liquid delivery mixture that provides an optimally adjusted pH for absorption of the active ingredients when administered to a patient.

In some embodiments, formulations can comprise all ingredients A-F, or only a subset of ingredients, in any combination (e.g., A through E, B through F, A+C+E+F, etc.). Ingredients A through F may be combined to form one exemplary formulation for non-invasive delivery of an unaltered active agent across a membrane. Certain compounds and/or compositions may be classified in multiple ingredient categories. Thus, one ingredient may be classified as an ingredient A and an ingredient F. The variations of the system allow for the prescribed variations of ingredients A to F within the complex in order to create optimal kinetics for the non-invasive delivery of pharmacologically-active agents through mammalian membranes, which in turn, allows for the success of a specific application based on clinical parameters.

As used herein, ingredients A and F are referred to as “vehicles” of the system. Certain embodiments include ingredients C and B in combination with either ingredient A or ingredient F. Preferred embodiments include at least ingredients A, F, and D. Some embodiments may include ingredients C, E, A and F. In preferred embodiments, ingredient A is vitamin B12, most preferably cobalamin. In another preferred embodiments, ingredient C is arginine. Preferred systems include at least one vehicle. More preferred systems include the metal-complexed vehicle in conjunction with the pH adjusting vehicle. Preferably, these ingredients interact with the active agent through ionic interactions. Ingredients B and D are optional.

For preferred embodiments, the procedure for the manufacture of the delivery system with the active agent(s) into a dry powder or film can simply include the mixing of ingredients A through F listed above, as would be understood to one skilled in the art. Preferably, the ingredients are mixed together, in accordance with procedures generally recognized as safe. Additionally, the procedure for the manufacture of the delivery system with the active agents(s) into a lotion may include mixing ingredients A through E above, wherein Ingredient F is a solution that is buffered and optimized for efficient transport of the ingredients across the mammalian membrane as would be understood to one skilled in the art.

Preferred embodiments include metal-complexed vehicles for the non-invasive transport of therapeutic active agents. In such embodiments, at least 1 microgram of an ionizable or ionized transition metal or metal-containing compound is added to an active agent to produce a non-invasive medicament. In a preferred embodiment, ingredient A is an ionizable or ionized transition metal or metal-containing compound that functions to transport the active agent across the membranes of the absorption site and stabilize the formulations during storage. Without being bound by theory, these ionizable or ionized transition metals or metal-containing compounds may readily change oxidation states as they move across the membranes in order to facilitate transport.

In other embodiments, Ingredient A may be combined with Ingredients B, C, D, and/or F. It is a surprising and unexpected discovery that mixtures of materials (e.g., Ingredients B, C, D and/or F) may facilitate the binding of the metal/metal-containing compounds (i.e., Ingredient A) to active agents (i.e., Ingredient E) for the efficient transport of physiological active agents across a membrane. It is also surprising and unexpected that certain formulations of the system comprising mixtures of materials (B, C, D and/or F) facilitate the optimal pH and ionic concentrations of the metal-complexed vehicles.

Metal-complexed vehicles complexed with therapeutic active agents showed similar efficacy with lower dosing than required with original modes of delivery (e.g., via intramuscular (IM) or subcutaneous (SC) injection, oral and/or nasal delivery) with reduced toxicities, which are likely related to the reduced levels of therapeutic active agents or decreased need for hepatic metabolism, or a combination thereof. That is, lower doses of active agents transported by metal-complexed vehicles show similar blood serum levels to the blood serum levels of active agents that were administered conventionally using much higher doses of the same active agent. Thus, formulations utilizing the metal-complexed vehicles are more efficient transporters of active agents than conventional modes of delivery.

Without being limited to theory, it is believed that metals taken in through the mouth naturally, rapidly directly absorb into the blood stream. Thus, a substance complexed to a metal and administered under the tongue, for example, would more efficiently pass through the sublingual membrane and into the blood without irreversible modifications. In contrast, if the metal-complexed vehicles are ingested, the metal-complexes would be digested. Digestive fluids and biological products would affect the active agents, which would be subject to degradation. The metal-complexes can be formed via ionic interactions. Preferably, the interactions between the metal-complexed vehicle and the active agent are ionic and reversible.

Any of the metals from 1A, 2A to 3B to 8B elements from the conventional Periodic Table of the Elements in various oxidation states can be used within the formulations disclosed herein. In particular embodiments, elements Scandium (SC), Titanium (Ti), Vanadium (V), Chromium (Cr), Manganese (Mn), Iron (Fe), Cobalt (Co), Nickel (Ni), Copper (Cu), Zinc (Zn), Gallium (Ga), Germanium (Ge), and Yttrium (Y), Zirconium (Zr), Niobium (Nb), Molybdenum (Mo), Technetium (Tc), Ruthenium (Ru), Rhodium (Rh), Palladium (Pd), Silver (Ag), Cadmium (Cd), Indium (In), Tin (Sn), Antimony (Sb), and Lanthanum (La), Hafnium (Hf), Tantalum (Ta), Tungsten (W,) Rhenium (Re), Iridium (Ir), Platinum (Pt), Gold (Au), Mercury (Hg), Thallium (Tl), Lead (Pb), Bismuth (Bi), Cerium (Ce), Praseodymium (Pr), Neodymium (Nd), Promethium (Pm), Samarium (Sm), Europium (Eu), Gadolinium (Gd), Terbium (Tb), Dysprosium (Dy), Holmium (Ho), Erbium (Er), Thulium (Tm), Ytterbium (Yb), Lutetium (Lu) can be utilized. Preferably, Cobalt is used.

The amount of ingredient A ranges from about 0 mg to about 1000 mg. In some embodiments, specific ingredient A dosages within a single formulation can include, but are not limited to, about 0.001, 0.005, 0.1, 0.2, 0.5, 1, 2, 5, 10, 25, 50, 75, 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, and 1000 mg. In other embodiments, Ingredient A can comprise, but is not limited to, from, about 0.01 to 95%, such as 0.01, 0.05, 0.1, 0.5, 1, 2, 5, 10, 20, 30, 40, 50, 60, 70, 80, 90 or about 95% of the total composition by weight.

Preferred embodiments may also include pH adjusting vehicles, such as Ingredient F, for the non-invasive transport of therapeutic active agents. In one embodiment, the formulation includes at least a solid or liquid delivery mixture that provides an optimally adjusted pH to facilitate the transport of active agents across the membrane by providing an acidic component and a basic component which provides a distinct (or adjustable) pH for the formulation. Absorption of active agents varies from patient to patient, and the optimal pH for absorption may accordingly vary. It would also be known to one skilled in the art the ideal, or practical, pH range for absorption of each active ingredient. The pH adjusting vehicle may preferably “sweep” the pH values from basic pH values to acidic pH values, or from acidic pH values to basic pH values. Preferably, the pH adjusting vehicle can be a mixture of solid, and in some cases liquid, components. The acidic components may be physically separated from the basic component until administration. When the acidic components and the basic components interact at the absorption site, the pH would preferably change from one pH value to another (e.g., 6.7 to 7.7 pH units) over a period of time (e.g., 1 to 180 seconds).

As mentioned above, Ingredient F can comprise both an acid component and a basic component within the same formulation in order to facilitate pH sweeping effects. To prevent reaction of the acidic and basic components prior to administration, one embodiment includes multiple, non-adjacent layers in the formulation. Alternatively, by designing an easily removable layer in the formulation to separate the acid and basic components, a reaction would be avoided or limited until a desired time. In some variations, the easily removable layer may be comprised of gelatinous or hydrophobic materials that prevent the mixing of the acidic component and basic component.

In other variations, it may be advantageous to “program” the dissolution of the formulation such that as the formulation dissolves, the formulation proceeds from acidic to basic pH values. Additionally, a dissolving tablet or other delivery vehicle may proceed from basic to acidic pH values. In either condition, the formulation will provide a pH range that optimizes the absorption of the drug or other active ingredient, thereby facilitating the rapid delivery of the therapeutic active agent, Preferably, the targeted pH corresponds to the optimal pH range of the active agent(s) of the formulation.

The mechanism of action for the pH sweep can preferably mitigate the effect of a membranes (or tissue's) normal pH that may present or otherwise impede absorption of the delivery systems. The pH sweep may further mitigate the effect of an active agent's pH that may also potentially impede absorption using the non-invasive delivery systems. The changes in pH value, as affected by the formulation, would provide rapid and preferably consistent delivery of the active agent.

Acidic components may include pharmaceutically acceptable excipients that can increase acidity. Acidic components used within formulations can include, but are not limited to, citric acid, acetic acid, tartaric acid, ascorbic acid, benzoic acid, erythorbic acid, fumaric acid, gluconic acid inosinic acid, lactic acid, malic acid, oxalic acid, pectic acid, phosphoric acid, sorbic acid, propionic acid, and potassium bitartarate, or any combination thereof. Additional examples include potassium bitartarate, sodium citrate/citric acid, phosphoric acid salts and mixtures, borax salts, 3-{[tris(hydroxymethyl)methyl]amino}propanesulfonic acid , N,N-bis(2-hydroxyethyl)glycine, tris(hydroxymethyl)methylamine, N-tris(hydroxymethyl)methylglycine, dimethylarsinic acid, organosulfonic acid derivatives, N,N-bis(2-hydroxyethyl)glycin, various amino acids, and other compounds that provide an ionic strength or a determined pH sweep range during absorption.

Preferably the amount of acid component corresponds to the amount of acidity required for, or corresponding to, the pH desired for a particular active agent. The amount may range from about 0.01 to about 1000 mg. Specific acidic dosages within a single formulation can include, but are not limited to, about 0.05, 1, 2, 5, 10, 25, 50, 75, 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, and about 1000 mg. The acidic component can comprise, but is not limited to, about 0.05 to 95% of the total composition by weight, such as 0.05, 0.1, 0.2, 0.5, 1, 2, 5, 10, 20, 30, 40, 50, 60, 70, 80, 90 or 95% of the total composition by weight. Preferably, the amount of Ingredient F is sufficient to effectively or sufficiently sweep the pH.

Basic components may include pharmaceutically acceptable excipients that can increase alkalinity. Basic components used within formulations can include, but are not limited to, sodium or potassium bicarbonate, sodium or potassium carbonate, cream of tartar, salts of acids listed above, organic amines, various metal salt carbonates, as well as chlorophyll and/or demetallized chlorophyll, or any combination thereof. Additional bases may include dipotassium tartarate, organic amines, pyridines, pyrimidines, pyradazines, quinazolines, quinoxalines, quinazolines, purines, and other nitrogen containing organic bases. In the case of the carbonates listed above, a slight effervescence action may be noted, which may help the patient realize that dissolution of the tablet is complete.

Preferably the amount of basic component corresponds to the amount of alkalinity required for the active agent. The amount may range from about 0.01 to about 1000 mg. Specific basic dosages within a single formulation can include, but are not limited to, about 0.05, 0.1, 0.5, 1, 2, 5, 10, 25, 50, 75, 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, and about 1000 mg. The basic component can comprise, but is not limited to, 0.1 to 95% of the total composition weight, such as about 0.01, 0.02, 0.5, 1, 2, 5, 10, 20, 30, 40, 50, 60, 70, 80, 90 or about 95% of the total composition by weight.

Preferably, Ingredient B, which is a salt that is ionized or readily ionizable may be added to formulations to provide stability to the formulation. More preferably, salts may aid in the development of ionic interactions between the various components of the formulations, thereby stabilizing the formulation. Preferably, salts may also protect the formulations from oxidation, and aid in preventing contamination from microbes such as bacteria, mold, or yeast. As a result, refrigeration or specialized environmental conditions for the formulations may not be required, the physiologic functions of the active agents can be maintained, and/or the formulations can have prolonged of shelf-life.

Salts of the present system include, but are not limited to, common salts that are used to provide ionically stable environments for efficient absorption of active agents. Specific salts would be known to one skilled in the art. Non-limiting examples may include sodium chloride, phosphoric acid salts, benzoic acid, citrate salts, tartaric acid salts, borax salts, 3-{[tris(hydroxymethyl)methyl]amino}propanesulfonic acid, N,N-bis(2-hydroxyethyl)glycine, tris(hydroxymethyl)methylamine, N-tris(hydroxymethyl)methylglycine, dimethylarsinic acid, organosulfonic acid derivatives, N,N-bis(2-hydroxyethyl)glycin, various amino acids, and other compounds that provide an ionic strength or a determined pH buffering range for stability of the active ingredients.

The ionic strength of the ingredients within the formulation can be adjusted in order to reproduce physiological ionic strengths of solutions in vivo in order to facilitate the absorption of the formulation across a membrane. As would be understood to a person of skill in the art, the ionic strength of a solution is a measure of the concentration of ions in that solution. Ionic compounds, when dissolved in water, dissociate into ions. Total electrolyte concentration in solution may affect the dissociation and/or the solubility of different salts. The ionic strength of the various ingredients can be calculated using known techniques, and the ingredients can subsequently be adjusted. For example, the formulation can be adjusted to have the same ionic strength as blood (about 0.154 NaCl). For example, the concentrations of salts (ingredient B) can be adjusted in order to provide the formulations disclosed herein with a desired ionic strength.

The amount of Ingredient B may range from about 0.01 to about 1000 mg, Specific Ingredient B dosages within a single formulation can include, but are not limited to, about 0.01, 0.02, 0.05, 0.1, 0.5, 1, 5, 10, 25, 50, 75, 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, and about 1000 mg. Ingredient B can comprise, but is not limited to, about 0.01 to 95% of the total composition weight, such as about 0.01, 0.02, 0.05, 0.1, 0.5, 1, 2, 5, 10, 20, 30, 40, 50, 60, 70, 80, 90 or about 95% of the total composition by weight.

Ingredient C, which is an amino acid, protein, sugar, and/or detergent, may be added to formulations to readily, but reversibly, bind to ingredients A and B. Preferably, Ingredient C may enhance the transport of the metal (i.e., Ingredient A) across absorption sites of the membrane. In preferred embodiments, Ingredient C is added to facilitate transport of the active agent. In some instances, compounds used in Ingredient A may be the same as compounds for Ingredient C. Examples of suitable proteins, sugars, and/or detergents in the present formulations include, but are not limited to, chlorophyllin, sodium glycodeoxycholate, chlorophyll, vitamin B12, amino acids, and the porphyrin ring structures such as found in both chlorophyll and vitamin B12. Preferably, vitamin B12 may be a synthetic or natural form, including but not limited to, cobolamine, cyanocobalamin, hydroxycobalamin, and/or methylcobalamin. Formulations may use a mixture of different forms of vitamin B12. Additionally, sodium glycodeoxycholate, and other biological detergent molecules may be of interest. Absorption through the skin and mucosal membranes may also be enhanced particularly when used with polar organic adjuvants such as methylsulfonylmethane (MSM) and/or dimethyl sulfoxide (DMSO).

Sugars may include natural or synthetic sugars. Sugars may also comprise monosaccharides, disaccharides or oligosaccharides. In some embodiments, sugars may be sugar alcohols. In other embodiments, sugars may include sugar substitutes. Preferably, a sugar may include glucose, xylose, xylitol, sorbitol, erythritol, mannitol, lactose, and fructose. In some embodiments 5 carbon sugars are used. In alternate embodiments, 6 carbon sugars are used. Sugars may be five- or six-ring sugars in embodiments

The amount of Ingredient C may range from about 0.01 to about 1000 mg. Specific Ingredient C dosages within a single formulation can include, but are not limited to, about 0.01, 0.02, 0.05, 0.1, 0.5, 1, 2, 5, 10, 25, 50, 75, 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, and about 1000 mg. Ingredient C can comprise, but is not limited to, about 2 to 95% of the total composition weight, such as about 0.01, 0.02, 0.05, 0.1, 0.5, 1, 2, 5, 10, 20, 30, 40, 50, 60, 70, 80, 90 or about 95% of the total composition by weight.

Ingredient D which is a buffered solution, film or powder, may be added to the formulation to optimize the pH of the mixture. Preferably, Ingredient D provides stability for the shelf life of the formulation. Examples of Ingredient D include, but are not limited to, potassium bitartarate, sodium citrate/citric acid, phosphoric acid salts and mixtures, polyhydric alcohols, and various parabens.

The amount of Ingredient D may range from about 0.01 to about 1000 mg. Specific Ingredient D dosages within a single formulation can include, but are not limited to, about 0.01, 0.02, 0.05, 0.1, 0.5, 1, 5, 10, 25, 50, 75, 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, and about 1000 mg. Ingredient D can comprise, but is not limited to, about 2 to 95% of the total composition weight, such as about 0.01, 0.02, 0.05, 0.1, 0.5, 1, 2, 5, 10, 20, 30, 40, 50, 60, 70, 80, 90 or about 95% of the total composition by weight.

Ingredient E includes the therapeutic active agent(s) transported across membranes via the system described herein. The pharmacological active agents can include any molecule or composition in any number or amount. Preferably, the active agent may be used individually or in combination with one another.

Classes of active agents can include analgesics (acetomenaphine (e.g., acetomenaphine, cox-1 and/or cox-2 inhibitors, ibuprofen, lidocaine), emergency medications (epinephrine, atropine, 17-(cyclopropylmethyl)-4,5α-epoxy-3,14-dihydroxymorphinan-6-one (naltrexone and flumazenil), anti-arrhythmics (amiodarone, diltiazem and atropine), lipid modulators (statins, atorvastatin); anti-convulsants (phenytoin sodium, topiramate and oxcarbazepine), anti-coagulants (low molecular weight heparin, enoxaparin), vitamins and nutritional supplements (co-enzyme Q10); corticosteroids (prednisone), oncology agents (paclitaxel and carboplatin), centrally active agents (sumatriptan), micropeptides (Xen 2174), rheumatologic agents (etanercept and adalimumab), bone marrow stimulators (filgrastim and epoetin alfa), osteoporosis medications (teriparatide), growth factors (somatotropin), immune modulators (gamma globulin), central and peripheral neuromuscular disorder agents (glatirameracetate), endocrine (human growth hormone (HGH), profasi (HCG analogue), insulin and/or insulin analogues), and vascular tone modulators (sildenafil). An additional example of active agents may include PT-141.

Exemplary active agents include agents for treating infections such as antibacterial, anti-fungal and antibiotic agents; for treating cardiovascular conditions such as chlorothiazide (diuretic), propranolol (antihypertensive), hydralazine (peripheral vasodilator), isosorbide or nitroglycerin (coronary vasodilators), metoprolol (beta-blocker), procainamide (antiarrythmic), clofibrate (cholesterol reducer) or coumadin (anticoagulant); agents for treating internal conditions such as conjugated estrogen (hormone), tolbutamide (antidiabetic), levothyroxine (thyroid conditions), propantheline (antispasmodic), cimetidine (antacid), phenyl propanolamine (anti-obesity), atropine or diphenoxalate (anti-diarrheal agents), docusate (laxative), or prochlorperazine (antinauseant); agents for treating mental health conditions such as haloperidol or chlorpromazine (tranquilizers), doxepin (psychostimulant), phenytoin (anticonvulsant), levo dopa (anti-parkinism), benzodiazepine (anti-anxiety) or phenobarbital (sedative); anti-inflammatory agents such as fluorometholone, acetaminophen, phenacetin, aspirin, hydrocortisone, or predisone; anti-histamines such as diphenhydramine hydrochloride or dexchlorpheniramine maleate; antibiotics such as sulfanilamide, sulfamethizole, tetracycline hydrochloride, penicillin and its derivatives, cephalosporin derivatives or erythromycin; chemotherapeutic agents such as sulfathiazole, doxorubicin, cisplatin or nitrofurazone; topical anaesthetics such as benzocaine; cardiac tonics such as digitalis or digoxin; antitussives and expectorants such as codeine phosphate, dextromethorphan or isoproterenol hydrochloride; oral antiseptics such as chlor hexidine hydrochloride or hexylresorcinol; enzymes such as lysozyme hydrochloride or dextronase; birth control agents such as estrogen; ophthalmic treating agents such as timolol or gentamycin, and the like. In addition, active agents may also include whole proteins such as the VP3 capsid protein (also known as the VPThr and VP1 capsid proteins in other nomenclature systems) as described in U.S. Pat. No. 4,140,763, which is incorporated herein in its entirety, insulin or interferon; polypeptide treating agents such as endorphins, human growth hormone, or bovine growth hormone, or still lower molecular weight polypeptides or conjugates of those polypeptides linked protein carriers.

The system can optionally further comprise an effective amount of active agents selected from at least one of an anti-infective, a cardiovascular system drug, a central nervous system drug, an autonomic nervous system drug, a respiratory tract drug, a gastrointestinal (GI) tract drug, a hormonal drug, a drug for fluid or electrolyte balance, a hematologic drug, an antineoplactic, an immunomodulation drug, an ophthalmic, otic or nasal drug, a topical drug, a nutritional drug, a statin, or the like.

Active agents can also be at least one selected from nonnarcotic analgesics or at least one selected from antipyretics, nonsteroidal anti-inflammatory drugs, narcotic or at least one opiod analgesics, sedative-hypnotics, anticonvulsants, antidepressants, antianxiety drugs, antipsychotics, central nervous system stimulants, antiparkinsonians, and miscellaneous central nervous system drugs. The active agent can be at least one selected from cholinergics (parasympathomimetics), anticholinergics, adrenergics (sympathomimetics), adrenergic blockers (sympatholytics), skeletal muscle relaxants, and neuromuscular blockers. Nonnarcotic analgesic or antipyretic can be at least one selected from acetaminophen, asprin, choline magnesium trisalicylate, diflunisal, and magnesium salicylate. Nonsteroidal anti-inflammatory drug can be at least one selected from celecoxib, diclofenac potassium, diclofenac sodium, etodolac, fenoprofen calcium, flurbiprofen, ibuprofen, indomethacin, ketoprofen, ketorolac tromethamine, nabumetone, naproxen, naproxen sodium, oxaprozin, piroxicam, rofecoxib, and sulindac. Narcotic or opiod analgesic can be at least one selected from alfentanil hydrochloride, buprenorphine hydrochloride, butorphanol tartrate, codeine phosphate, codeine sulfate, fentanyl citrate, fentanyl transdermal system, fentanyl transmucosal, hydromorphone hydrochloride, meperidine hydrochloride, methadone hydrochloride, morphine hydrochloride, morphine sulfate, morphine tartrate, nalbophine hydrochloride, oxycodone hydrochloride, oxycodone pectinate, oxymorphone hydrochloride, pentazocine hydrochloride, pentazocine hydrochloride and naloxone hydrochloride, pentazocine lactate, propoxyphene hydrochloride, propoxyphene napsylate, remifentanil hydrochloride, sufentanil citrate, and tramadol hydrochloride. The sedative-hypnotic can be at least one selected from chloral hydrate, estazolam, flurazepam hydrochloride, pentobarbital, pentobarbital sodium, phenobarbital sodium, secobarbital sodium, temazopam, triazolam, zaleplon, and zolpidem tartrate.

The anticonvulsant can be at least one selected from acetazolamide sodium, carbamazepine, clonazepam, clorazepate dipotassium, diazepam, divalproex sodium, ethosuximde, fosphenytoin sodium, gabapentin, lamotrigine, magnesium sulfate, phenobarbital, phenobarbital sodium, phenytoin, phenytoin sodium, phenytoin sodium (extended), primidone, tiagabine hydrochloride, topiramate, valproate sodium, and valproic acid.

The antidepressant can be at least one selected from amitriptyline hydrochloride, amitriptyline pamoate, amoxapine, bupropion hydrochloride, citalopram hydrobromide, clomipramine hydrochloride, desipramine hydrochloride, doxepin hydrochloride, fluoxetine hydrochloride, imipramine hydrochloride, imipramine pamoate, mirtazapine, nefazodone hydrochloride, nortriptyline hydrochloride, paroxetine hydrochloride, phenelzine sulfate, sertraline hydrochloride, tranylcypromine sulfate, trimipramine maleate, and venlafaxine hydrochloride. The antianxiety drug can be at least one selected from alprazolam, buspirone hydrochloride, chlordiazepoxide, chlordiazepoxide hydrochloride, clorazepate dipotassium, diazepam, doxopin hydrochloride, hydroxyzine embonate, hydroxyzine hydrochloride, hydroxyzine pamoate, lorazepam, mephrobamate, midazolam hydrochloride, and oxazopam. The antipsychotic drug can be at least one selected from chlorpromazine hydrochloride, clozapine, fluphenazine decanoate, fluephenazine enanthate, fluphenazine hydrochloride, haloperidol, haloperidol decanoate, haloperidol lactate, loxapine hydrochloride, loxapine succinate, mesoridazine besylate, molindone hydrochloride, olanzapine, perphenazine, pimozide, prochlorperazine, quetiapine fumarate, risperidone, thioridazine hydrochloride, thiothixene, thiothixene hydrochloride, and trifluoperazine hydrochloride. The central nervous system stimulant can be at least one selected from amphetamine sulfate, caffeine, dextroamphetamine sulfate, doxapram hydrochloride, methamphetamine hydrochloride, methylphenidate hydrochloride, modafinil, pemoline, and phentermine hydrochloride. The anti-parkinsonian can be at least one selected from amantadine hydrochloride, benztropine mesylate, biperiden hydrochloride, biperiden lactate, bromocriptine mesylate, carbidopa-levodopa, entacapone, levodopa, pergolide mesylate, pramipexole dibydrochloride, ropinirole hydrochloride, selegiline hydrochloride, tolcapone, and trihexyphenidyl hydrochloride. The central nervous system active agent may be at least one selected from riluzole, bupropion hydrochloride, donepezil hydrochloride, droperidol, fluvoxamine maleate, lithium carbonate, lithium citrate, naratriptan hydrochloride, nicotine polacrilex, nicotine transdermal system, propofol, rizatriptan benzoate, sibutramine hydrochloride monohydrate, sumatriptan succinate, tacrine hydrochloride, and zolmitriptan. The cholinergic (e.g., parasymathomimetic) active agent may be at least one selected from bethanechol chloride, edrophonium chloride, neostigmine bromide, neostigmine, methylsulfate, physostigmine salicylate, and pyridostigmine bromide. The anticholinergics may be at least one selected from atropine sulfate, dicyclomine hydrochloride, glycopyrrolate, hyoscyamine, hyoscyamine sulfate, propantheline bromide, scopolamine, scopolamine butylbromide, and scopolamine hydrobromide. The adrenergic (sympathomimetics) active agent may be at least one selected from dobutamine hydrochloride, dopamine hydrochloride, metaraminol bitartrate, norepinephrine bitartrate, phenylephrine hydrochloride, pseudoephedrine hydrochloride, and pseudoephedrine sulfate. The adrenergic blocker 1 (sympatholytic) may be at least one selected from dibydroergotamine mesylate, ergotamine tartrate, methysergide maleate, and propranolol hydrochloride. The skeletal muscle relaxant may be at least one selected from baclofen, carisoprodol, chlorzoxazone, cyclobenzaprine hydrochloride, dantrolene sodium, methocarbamol, and tizanidine hydrochloride.

Neuromuscular blocker active agents can be at least one selected from atracurium besylate, cisatracurium besylate, doxacurium chloride, mivacurium chloride, pancuronium bromide, pipecuronium bromide, rapacuronium bromide, rocuronium bromide, succinylcholine chloride, tubocurarine chloride, and vecuronium bromide. The anti-infective active agent can be at least one selected from amebicides or at least one antiprotozoals, anthelmintics, antifungals, antimalarials, antituberculotics or at least one antileprotics, aminoglycosides, penicillins, cephalosporins, tetracyclines, sulfonamides, fluoroquinolones, antivirals, macrolide anti-infectives, and miscellaneous anti-infectives. The cardiovascular active agent can be at least one selected from inotropics, antiarrhythmics, antianginals, antihypertensives, antilipemics, and miscellaneous cardiovascular drugs. The central nervous system active agent can be at least one selected from nonnarcotic analgesics or at least one selected from antipyretics, nonsteroidal anti-inflammatory drugs, narcotic or at least one opioid analgesics, sedative hypnotics, anticonvulsants, antidepressants, antianxiety drugs, antipsychotics, central nervous system stimulants, antiparkinsonians, and miscellaneous central nervous system drugs. The autonomic nervous system drug can be at least one selected from cholinergics (parasympathomimetics), anticholinergics, adrenergics (sympathomimetics), adrenergic blockers (sympatholytics), skeletal muscle relaxants, neuromuscular blockers. The respiratory tract active agent may be at least one selected from antihistamines, bronchodilators, expectorants or at least one antitussive, and miscellaneous respiratory drugs. The GI tract active agent may be at least one selected from antacids or at least one adsorbents or at least one antiflatulents, digestive enzymes or at least one gallstone solubilizers, antidiarrheals, laxatives, antiemetics, and antiulcer drugs. The hormonal active agent may be at least one selected from corticosteroids, androgens or at least one anabolic steroids, estrogens or at least one progestins, gonadotropins, antidiabetic drugs or at least one glucagon, thyroid hormones, thyroid hormone antagonists, pituitary hormones, and parathyroid-like drugs. The active agent for fluid and electrolyte balance can be at least one selected from diuretics, electrolytes or at least one replacement solution, acidifiers or at least one alkalinizer. The hematologic active agent may be at least one selected from hematinics, anticoagulants, blood derivatives, thrombolytic enzymes. The antineoplastics can be at least one selected from alkylating drugs, antimetabolites, antibiotic antineoplastics, antineoplastics that alter hormone balance, and miscellaneous antineoplastics. The immunomodulation active agent may be at least one selected from immunosuppressants, vaccines or at least one toxoid, antitoxins or at least one antivenins, immune serums, biological response modifiers. The ophthalmic, otic, and nasal active agent may be at least one selected from ophthalmic anti-infectives, ophthalmic anti-inflammatories, miotics, mydriatics, ophthalmic vasoconstrictors, miscellaneous ophthalmics, otics, and nasal active agents. The topical active agent can be at least one selected from local anti-infectives, scabicides or at least one pediculicides, and topical corticosteroids.

Amebicide or antiprotozoal can be at least one selected from atovaquone, chloroquine hydrochloride, chloroquine phosphate, metronidazole, metronidazole hydrochloride, and pentamidine isethionate. Anthelmintic active agents can be at least one selected from mebendazole, pyrantel pamoate, and thiabendazole. Antifungals may be at least one selected from amphotericin B. amphotericin B cholesteryl sulfate complex, amphotericin B lipid complex, amphotericin B liposomal, fluconazole, flucytosine, griseofulvin microsize, griseofulvin ultramicrosize, itraconazole, ketoconazole, nystatin, and terbinafine hydrochloride. The at least one antimalarial may be at least one selected from chloroquine hydrochloride, chloroquine phosphate, doxycycline, hydroxychloroquine sulfate, mefloquine hydrochloride, primaquine phosphate, pyrimethamine, and pyrimethamine with sulfadoxine. The at least one antituberculotic or antileprotic can be at least one selected from clofazimine, cycloserine, dapsone, ethambutol hydrochloride, isoniazid, pyrazinamide, rifabutin, rifampin, rifapentine, and streptomycin sulfate. The at least one aminoglycoside may be at least one selected from amikacin sulfate, gentanicin sulfate, neomycin sulfate, streptomycin sulfate, and tobramycin sulfate. The at least one penicillin may be at least one selected from amoxcillin/clavulanate potassium, amoxicillin trihydrate, ampicillin, ampicillin sodium, ampicillin tribydrate, ampicillin sodium/sulbactam sodium, cloxacillin sodium, dicloxacillin sodium, mezlocillin sodium, nafcillin sodium, oxacillin sodium, penicillin G benzathine, penicillin G potassium, penicillin G procaine, penicillin G sodium, and penicillin V potassium. Cephalosporin can be at least one selected from at least one of cefaclor, cefadroxil, cefazolin sodium, cefdinir, cefepime hydrochloride, cefixime, cefmetazole sodium, cefonicid sodium, cefoperazone sodium, cefotaxime sodium, cefotetan disodium, cefoxitin sodium, cefpodoxime proxetil, cefprozil, ceftazidime, ceftibuten, ceftizoxime sodium, ceftriaxone sodium, ceffiroxime axetil, cefuroxime sodium, cephalexin hydrocllloride, cephalexin monohydrate, cephradine, and loracarbef. Tetracycline can be at least one selected from demeclocycline hydrochloride, doxycycline calcium, doxycycline hyclate, doxycycline hydrochloride, doxycycline monobydrate, minocycline hydrochloride, and tetracycline hydrochloride. Sulfonamide can be at least one selected from co-trimoxazole, sulfadiazine, sulfamethoxazole, sulfisoxazole, and sulosoxazole acetyl. Fluoroquinolone can be at least one selected from alatrofloxacin mesylate, ciprofloxacin, enoxacin, levofloxacin, lomefloxacin hydrochloride, nalidixic acid, norfloxacin, ofloxacin, sparfloxacin, and trovafloxacin mesylate. Fluoroquinolone can be at least one selected from alatrofloxacin mesylate, ciprofloxacin, enoxacin, levofloxacin, lomefloxacin hydrochloride, nalidixic acid, norfloxacin, ofloxacin, sparfloxacin, and trovafloxacin mesylate. The antiviral active agent may be at least one selected from abacavir sulfate, acyclovir sodium, amantadine hydrochloride, amprenavir, cidofovir, delavirdine mesylate, didanosine, efavirenz, famciclovir, fomivirsen sodium, foscarnet sodium, ganciclovir, indinavir sulfate, lamivudine, lamivodine/zidovodine, nelfinavir mesylate, nevirapine, oseltamivir phosphate, ribavirin, rimantadine hydrochloride, ritonavir, saquinavir, saquinavir mesylate, stavodine, valacyclovir hydrochloride, zalcitabine, zanamivir, and zidovudine.

Macroline anti-infective active agents may be at least one selected from azithromycin, clarithromycin, dirithromycin, erythromycin base, erythromycin estolate, erythromycin ethylsuccinate, erythromycin lactobionate, and erythromycin stearate. Anti-infective active agents may also be at least one selected from aztreonam, bacitracin, chloramphenicol sodium sucinate, clindamycin hydrochloride, clindamycin palmitate hydrochloride, clindamycin phosphate, imipenem and cilastatin sodium, meropenem, nitrofurantoin macrocrystals, nitrofurantoin microcrystals, quinupristin/dalfopristin, spectinomycin hydrochloride, trimethoprim, and vancomycin hydrochloride. Inotropic active agent may be at least one selected from amrinone lactate, digoxin, and milrinone lactate. Antiarrhythmic active agents can be at least one selected from adenosine, amiodarone hydrochloride, atropine sulfate, bretylium tosylate, diltiazem hydrochloride, disopyramide, disopyramide phosphate, esmolol hydrochloride, flecainide acetate, ibutilide fumarate, lidocaine hydrochloride, mexiletine hydrochloride, moricizine hydrochloride, phenytoin, phenytoin sodium, procainamide hydrochloride, propafenone hydrochloride, propranolol hydrochloride, quinidine bisulfate, quinidine gluconate, quinidine polygalacturonate, quinidine sulfate, sotalol, tocainide hydrochloride, and verapamil hydrochloride.

Antianginal active agents may be at least one selected from amlodipidine besylate, amyl nitrite, bepridil hydrochloride, diltiazem hydrochloride, isosorbide dinitrate, isosorbide mononitrate, nadolol, nicardipine hydrochloride, nifedipine, nitroglycerin, propranolol hydrochloride, verapamil, and verapamil hydrochloride. Antihypertensive active agents may be at least one selected from acebutolol hydrochloride, amlodipine besylate, atenolol, benazepril hydrochloride, betaxolol hydrochloride, bisoprolol fumarate, candesartan cilexetil, captopril, carteolol hydrochloride, carvedilol, clonidine, clonidine hydrochloride, diazoxide, diltiazem hydrochloride, doxazosin mesylate, enalaprilat, enalapril maleate, eprosartan mesylate, felodipine, fenoldopam mesylate, fosinopril sodium, guanabenz acetate, guanadrel sulfate, guanfacine hydrochloride, hydralazine hydrochloride, irbesartan, isradipine, labetalol hydrchloride, lisinopril, losartan potassium, methyldopa, methyldopate hydrochloride, metoprolol succinate, metoprolol tartrate, minoxidil, moexipril hydrochloride, nadolol, nicardipine hydrochloride, nifedipine, nisoldipine, nitroprusside sodium, penbutolol sulfate, perindopril erbumine, phentolamine mesylate, pindolol, prazosin hydrochloride, propranolol hydrochloride, quinapril hydrochloride, ramipril, telmisartan, terazosin hydrochloride, timolol maleate, trandolapril, valsartan, and verapamil hydrochloride. Antilipemic active agents may be at least one selected from atorvastatin calcium, cerivastatin sodium, cholestyramine, colestipol hydrochloride, fenofibrate (micronized), fluvastatin sodium, gemfibrozil, lovastatin, niacin, pravastatin sodium, simvastatin. Cardiovascular active agents may be at least one selected from abciximab, alprostadil, arbutamine hydrochloride, cilostazol, clopidogrel bisulfate, dipyridamole, eptifibatide, midodrine hydrochloride, pentoxifylline, ticlopidine hydrochloride, and tirofiban hydrochloride. Antihistamine active agents may be at least one selected from brompheniramine maleate, cetirizine hydrochloride, chlorpheniramine maleate, clemastine fumarate, cyproheptadine hydrochloride, diphenLydramine hydrochloride, fexofenadine hydrochloride, loratadine, promethazine hydrochloride, promethazine theoclate, and triprolidine hydrochloride.

Bronchodilators may be at least one selected from albuterol, albuterol sulfate, aminophylline, atropine sulfate, ephedrine sulfate, epinephrine, epinephrine bitartrate, epinephrine hydrochloride, ipratropium bromide, isoproterenol, isoproterenol hydrochloride, isoproterenol sulfate, levalbuterol hydrochloride, metaproterenol sulfate, oxtriphylline, pirbuterol acetate, salmeterol xin.afoate, terbutaline sulfate, and theophylline. Expectorants or antitussives may be at least one selected from benzonatate, codeine phosphate, codeine sulfate, dextramethorphan hydrobromide, diphenhydramine hydrochloride, guaifenesin, and hydromorphone hydrochloride. Respiratory active agent may be at least one selected from acetylcysteine, beclomethasone dipropionate, beractant, budesonide, calfactant, cromolyn sodium, dornase alfa, epoprostenol sodium, flunisolide, palivizumab, triamcinolone acetonide, zafirlukast, and zileuton. Antacids, adsorbents, or antiflatulents may be at least one selected from aluminum carbonate, aluminum hydroxide, calcium carbonate, magaldrate, magnesium hydroxide, magnesium oxide, simethicone, and sodium bicarbonate. Digestive enymes or gallstone solubilizer active agents can be at least one selected from pancreatin, pancrelipase, and ursodiol. Antidiarrheal active agents can be at least one selected from attapulgite, bismuth subsalicylate, calcium polycarbophil, diphenoxylate hydrochloride or atropine sulfate, loperamide, octreotide acetate, opium tincture, and opium tincure (camphorated). Laxative active agents may be at least one selected from bisocodyl, calcium polycarbophil, cascara sagrada, cascara sagrada aromatic fluidextract, cascara sagrada fluidextract, castor oil, docusate calcium, docusate sodium, glycerin, lactulose, magnesium citrate, magnesium hydroxide, magnesium sulfate, methylcellulose, mineral oil, polyethylene glycol or electrolyte solution, psyllium, senna, and sodium phosphates. Antiemetic active agents may be at least one selected from chlorpromazine hydrochloride, dimenhydrinate, dolasetron mesylate, dronabinol, granisetron hydrochloride, meclizine hydrochloride, metocloproamide hydrochloride, ondansetron hydrochloride, perphenazine, prochlorperazine, prochlorperazine edisylate, prochlorperazine maleate, promethazine hydrochloride, scopolamine, thiethylperazine maleate, and trimethobenzamide hydrochloride. Antiulcer acive agents may be at least one selected from cimetidine, cimetidine hydrochloride, famotidine, lansoprazole, misoprostol, nizatidine, omeprazole, rabeprozole sodium, rantidine bismuth citrate, ranitidine hydrochloride, and sucralfate.

Coricosteroid active agents may be at least one selected from betamethasone, betamethasone acetate or betamethasone sodium phosphate, betamethasone sodium phosphate, cortisone acetate, dexamethasone, dexamethasone acetate, dexamethasone sodium phosphate, fludrocortisone acetate, hydrocortisone, hydrocortisone acetate, hydrocortisone cypionate, hydrocortisone sodium phosphate, hydrocortisone sodium succinate, methylprednisolone, methylprednisolone acetate, methylprednisolone sodium succinate, prednisolone, prednisolone acetate, prednisolone sodium phosphate, prednisolone tebutate, prednisone, triamcinolone, triamcinolone acetonide, and triamcinolone diacetate.

Androgen or anabolic steroids may be at least one selected from danazol, fluoxymesterone, methyltestosterone, nandrolone decanoate, nandrolone phenpropionate, testosterone, testosterone cypionate, testosterone enanthate, testosterone propionate, and testosterone transdermal system. Estrogen or progestin may be at least one selected from esterified estrogens, estradiol, estradiol cypionate, estradiol/norethindrone acetate transdermal system, estradiol valerate, estrogens (conjugated), estropipate, ethinyl estradiol, ethinyl estradiol and desogestrel, ethinyl estradiol and ethynodiol diacetate, ethinyl estradiol and desogestrel, ethinyl estradiol and ethynodiol diacetate, ethinyl estradiol and levonorgestrel, ethinyl estradiol and norethindrone, ethinyl estradiol and norethindrone acetate, ethinyl estradiol and norgestimate, ethinyl estradiol and norgestrel, ethinyl estradiol and norethindrone and acetate and ferrous fumarate, levonorgestrel, medroxyprogesterone acetate, mestranol and norethindron, norethindrone, norethindrone acetate, norgestrel, and progesterone. Gonadroptropin active agents may be at least one selected from ganirelix acetate, gonadoreline acetate, histrelin acetate, and menotropins. Antidiabetic active agents may be at least one selected from acarbose, chlorpropamide, glimepiride, glipizide, glucagon, glyburide, insulins, metformin hydrochloride, miglitol, pioglitazone hydrochloride, repaglinide, rosiglitazone maleate, and troglitazone. Thyroid hormone active agents may be at least one selected from levothyroxine sodium, liothyronine sodium, liotrix, and thyroid. Thyroid hormone antagonist active agents may be at least one selected from methimazole, potassium iodide, potassium iodide (saturated solution), propylthiouracil, radioactive iodine (sodium iodide), and strong iodine solution. Pituitary hormone active agents may be at least one selected from corticotropin, cosyntropin, desmophressin acetate, leuprolide acetate, repository corticotropin, somatrem, somatropin, and vasopressin. Parathyroid-like active agents may be at least one selected from calcifediol, calcitonin (human), calcitonin (salmon), calcitriol, dihydrotachysterol, and etidronate disodium. Diuretics can be at least one selected from acetazolamide, acetazolamide sodium, amiloride hydrochloride, bumetanide, chlorthalidone, ethacrynate sodium, ethacrynic acid, furosemide, hydrochlorothiazide, indapamide, mannitol, metolazone, spironolactone, torsemide, triamterene, and urea. Electrolyte or replacement solution active agents can be at least one selected from calcium acetate, calcium carbonate, calcium chloride, calcium citrate, calcium glubionate, calcium gluceptate, calcium gluconate, calcium lactate, calcium phosphate (dibasic), calcium phosphate (tribasic), dextran (high-molecular-weight), dextran (low-molecular-weight), hetastarch, magnesium chloride, magnesium sulfate, potassium acetate, potassium bicarbonate, potassium chloride, potassium gluconate, Ringer's injection, Ringer's injection (lactated), and sodium chloride.

Hematinic active agents may be at least one selected from ferrous fumarate, ferrous gluconate, ferrous sulfate, ferrous sulfate (dried), iron dextran, iron sorbitol, polysaccharide iron complex, sodium ferric gluconate complex. Anticoagulant active agents may be at least one selected from ardeparin sodium, dalteparin sodium, danaparoid sodium, enoxaparin sodium, heparin calcium, heparin sodium, and warfarin sodium. One blood derivative can be at least one selected from albumin 5%, albumin 25%, antihemophilic factor, anti inhibitor coagulant complex, antithrombin m (human), factor IX (human), factor IX complex, and plasma protein fractions. Thrombolytic enzyme active agents may be selected from alteplase, anistreplase, reteplase (recombinant), streptokinase, urokinase. Alkylating active agents may be at least one selected from busulfan, carboplatin, carmustine, chlorambucil, cisplatin, cyclophosphamide, ifosfamide, lomustine, mechl oreth amine hydrochloride, melphalan, melphal an hydrochloride, streptozocin, temozolomide, thiotepa. Antimetabolite can be selected from capecitabine, cladribine, cytarabine, floxuridine, fludarabine phosphate, fluorouracil, hydroxyurea, mercaptopurine, methotrexate, methotrexate sodium, thioguanine. Antibiotic antineoplastic can be selected from bleomycin sulfate, dactinomycin, daunorubicin citrate liposomal, daunorubicin hydrochloride, doxorubicin hydrochloride, doxorubicin hydrochloride liposomal, epirubicin hydrochloride, idaubicin hydrochloride, mitomycin, pentostatin, plicamycin, and valrubicin. Antineoplastics may be selected from anastrozole, bicalutamide, estramustine phosphate sodium, exemestane, flutamide, goserelin acetate, letrozole, leuprolide acetate, megestrol acetate, nilutamide, tamoxifen citrate, testolactone, toremifene citrate, asparaginase, bacillus Calmette-Guerin (BCG), dacarbazine, docetaxel, etoposide, etoposide phosphate, gemcitabine hydrochloride, irinotecan hydrochloride, mitotane, mitoxantrone hydrochloride, paclitaxel, pegaspargase, porfimer sodium, procarbazine hydrochloride, rituximab, teniposide, topotecan hydrochloride, trastuzumab, tretinoin, vinblastine sulfate, vincristine sulfate, and vinorelbine tartrate. Immunosuppressant active agents may be at least one selected from azathioprine, basiliximab, cyclosporine, daclizumab, lymphocyte immune globulin, muromonab-CD3, mycophenolate mofetil, mycophenolate mofetil hydrochloride, sirolimus, and tacrolimus. Vaccine or tosoid active agents may be at least one selected from BCG vaccine, cholera vaccine, diphtheria and tetanus toxoids (adsorbed), diphtheria and tetanus toxoids and acellular pertussis vaccine adsorbed, diphtheria and tetanus toxoids and whole-cell pertussis vaccine, Haemophilius b conjugate vaccines, hepatitis A vaccine (inactivated), hepatisis B vaccine (recombinant), influenza virus vaccine 1999-2000 trivalent types A & B (purified surface antigen), influenza virus vaccine 1999-2000 trivalent types A & B (subvirion or purified subvirion), influenza virus vaccine 1999-2000 trivalent types A & B (whole virion), Japanese encephalitis virus vaccine (inactivated), influenza H1N1 vaccine, Lymes disease vaccine (recombinant OspA), measles and mumps and rubella virus vaccine (live), measles and mumps and rubella virus vaccine (live attenuated), measles virus vaccine (live attenuated), meningococcal polysaccharide vaccine, mumps virus vaccine (live), plague vaccine, pneumococcal vaccine (polyvalent), poliovirus vaccine (inactivated), poliovirus vaccine (live, oral, trivalent), rabies vaccine (adsorbed), rabies vaccine (human diploid cell), rubella and mumps virus vaccine (live), rubella virus vaccine (live, attenuated), tetanus toxoid (adsorbed), tetanus toxoid (fluid), typhoid vaccine (oral), typhoid vaccine (parenteral), typhoid Vi polysaccharide vaccine, varicella virus vaccine, and yellow fever vaccine. Antitoxin or antivenin (or antivenom) active agents may be at least one selected from black widow spider antivenin, Crotalidae antivenom (polyvalent), diphtheria antitoxin (equine), and Micrurus fulvius antivenin). Immune serum active agents may be at least one selected from cytomegalovirus immune globulin, hepatitis B immune globulin (human), immune globulin intramuscular, immune globulin intravenous, rabies immune globulin (human), respiratory syncytial virus immune globulin intravenous (human), Rho(D) immune globulin (human), Rho(D) immune globulin intravenous (human), tetanus immune globulin (human), and varicella-zoster immune globulin. Biological response modifiers can be at least one selected from aldesleukin, epoetin alfa, filgrastim, glatiramer acetate for injection, interferon alfacon-1, interferon alfa-2a (recombinant), interferon alfa-2b (recombinant), interferon beta-1a, interferon beta-1b (recombinant), interferon gamma-1b, levamisole hydrochloride, oprelvekin, and sargramostim. Ophthalmic anti-infectives may be selected form bacitracin, chloramphenicol, ciprofloxacin hydrochloride, erythromycin, gentamicin sulfate, ofloxacin 0.3%, polymyxin B sulfate, sulfacetamide sodium 10%, sulfacetamide sodium 15%, sulfacetamide sodium 30%, tobramycin, vidarabine. Ophthalmic anti-inflammatory active agents may be at least one selected from dexamethasone, dexamethasone sodium phosphate, diclofenac sodium 0.1%, fluorometholone, flurbiprofen sodium, ketorolac tromethamine, prednisolone acetate, and prednisolone sodium phosphate.

Miotics may be at least one selected from acetylocholine chloride, carbachol (intraocular), carbachol (topical), echothiophate iodide, pilocarpine, pilocarpine hydrochloride, and pilocarpine nitrate. Mydriatic active agents may be at least one selected from atropine sulfate, cyclopentolate hydrochloride, epinephrine hydrochloride, epinephryl borate, homatropine hydrobromide, phenylephrine hydrochloride, scopolamine hydrobromide, and tropicamide. Ophthalmic vasoconstrictors may be at least one selected from naphazoline hydrochloride, oxymetazoline hydrochloride, and tetrahydrozoline hydrochloride. Ophthalmics can be at least one selected from apraclonidine hydrochloride, betaxolol hydrochloride, brimonidine tartrate, carteolol hydrochloride, dipivefrin hydrochloride, dorzolamide hydrochloride, emedastine difumarate, fluorescein sodium, ketotifen fumarate, latanoprost, levobunolol hydrochloride, metipranolol hydrochloride, sodium chloride (hypertonic), and timolol maleate. Otic (ear) active agents may be at least one selected from boric acid, carbamide peroxide, chloramphenicol, and triethanolamine polypeptide oleate-condensate. Nasal active agents may be at least one selected from beclomethasone dipropionate, budesonide, ephedrine sulfate, epinephrine hydrochloride, flunisolide, fluticasone propionate, naphazoline hydrochloride, oxymetazoline hydrochloride, phenylephrine hydrochloride, tetrabydrozoline hydrochloride, triamcinolone acetonide, and xylometazoline hydrochloride. Anti-infectives may also be at least one selected from acyclovir, amphotericin B., azelaic acid cream, bacitracin, butoconazole nitrate, clindamycin phosphate, clotrimazole, econazole nitrate, erythromycin, gentamicin sulfate, ketoconazole, mafenide acetate, metronidazole (topical), miconazole nitrate, mupirocin, naftifine hydrochloride, neomycin sulfate, nitrofurazone, nystatin, silver sulfadiazine, terbinafine hydrochloride, terconazole, tetracycline hydrochloride, tioconazole, and tolnaftate. Scabicide or pediculicide active agents may be at least one selected from crotamiton, lindane, permethrin, and pyrethrins. Corticosteroid may be at least one selected from betamethasone dipropionate, betamethasone valerate, clobetasol propionate, desonide, desoximetasone, diacetate, fluocinolone acetonide, fluocinonide, flurandrenolide, fluticasone propionate, halcionide, hydrocortisone, hydrocortisone acetate, hydrocortisone butyrate, hydrocorisone valerate, mometasone furoate, and triamcinolone acetonide. Other additional active agents, or classes of active agents include Tumor necrosis factor (TNF) antagonists (e.g., but not limited to a TNF chemical or protein antagonist, TNF monoclonal or polyclonal antibody or fragment, a soluble TNF receptor (e.g., p55, p70 or p85) or fragment, fusion polypeptides thereof, or a small molecule TNF antagonist, e.g., TNF binding protein I or II (TBP-1 or TBP-II), nerelimonmab, infliximab, enteracept, CDP-571, 2 5 CDP-870, afelimomab, lenercept, and the like). Active agents may further include an antirheumatic (e.g., methotrexate, auranofin, aurothioglucose, azathioprine, etanercept, gold sodium thiomalate, hydroxychloroquine sulfate, leflunomide, sulfasalzine), a muscle relaxant, a narcotic, a non-steroid anti-inflammatory drug (NSAID), an analgesic, an anesthetic, a sedative, a local anesthetic, a neuromuscular blocker, an antimicrobial (e.g., aminoglycoside, an antifungal, an antiparasitic, an antiviral, a carbapenem, cephalosporin, a flurorquinolone, a macrolide, a penicillin, a sulfonamide, a tetracycline, another antimicrobial), an antipsoriatic, a corticosteriod, an anabolic steroid, a diabetes related agent, a mineral, a nutritional, a thyroid agent, a vitamin, a calcium related hormone, an antidiarrheal, an antitussive, an antiemetic, an antiulcer, a laxative, an anticoagulant, an erythropieitin (e.g., epoetin alpha), a filgrastim (e.g., G-CSF, Neupogen), a sargramostim (GM 3 5 CSF, Leukine), an immunization, an immunoglobulin, an immunosuppressive (e.g., basiliximab, cyclosporine, daclizumab), a growth hormone, a hormone replacement drug, an estrogen receptor modulator, a mydriatic, a cycloplegic, an alkylating agent, an antimetabolite, a mitotic inhibitor, a radiopharmaceutical, an antidepressant, antimanic agent, an antipsychotic, an anxiolytic, a hypnotic, a sympathomimetic, a stimulant, donepezil, tacrrne, an asthma medication, a beta agonist, an inhaled steroid, a leukotriene inhibitor, a methylxanthine, a cromolyn, an epinephrine or analog, domase alpha, a cytokine or a cytokine antagonist. Non-limiting examples of such cytokines include, but are not limited to, any of the interleukins, including IL-1 to IL-23.

The amount of Ingredient E may range from about 0.01 to about 1000 mg. Specific Ingredient E dosages within a single formulation can include, but are not limited to, about 0.01, 0.02, 0.05, 0.1, 0.5, 1, 2, 5, 10, 25, 50, 75, 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, and about 1000 mg. Ingredient E can comprise, but is not limited to, about 0.01 to 95% of the total composition weight, such as about 0.01, 0.02, 0.05, 0.1, 0.5, 1, 2, 5, 10, 20, 30, 40, 50, 60, 70, 80, 90 or about 95% of the total composition by weight.

The formulations of the delivery system described herein may be in any suitable shape, size or form. The methods of formulating the formulations of the system may be found in such standard references as Remington: The Science and Practice of Pharmacy, A. Gennaro, ed., 20th edition, Lippincott, Williams & Wilkins, Philadelphia, Pa., which is herein incorporated by reference in its entirety. In general, the therapeutic formulations may range from less than about 0.01 grams to more than about 100 grams, preferably between about 0.01 g and about 10 g and more preferably between about 0.01 g and about 1 g.

The formulations described herein may be in the form of tablets (sustained-release, controlled-release, quick dissolve, multi-layer, bi-layer, etc.), pills, capsules, microcapsules, caplets, films, patches, topicals, lotions, gels, moisturizing creams, sun-screens, after-sun creams, anti-aging creams, ointments, liquids, powders, syrups, elixirs, suppositories, douches, pessaries, suspensions, solutions, liposomes, micelles, microparticles, nanoparticles, aerosols, inhalants, implants or other suitable formulations known to the art. Additional examples of these forms might include chachets, lollipops, sucking candies or lozenges, sprays, hydrogels, creams, emoliants, dissolvable wafers, rectal suppository, topical paints, internasal aerosols or dry powders.

These forms may be used as stand-alones or inserted into separate medical devices or protective biomembranes or devices, examples of which might include cuticle or nail paint, cosmetic applications including exfoliants, debriding, chemical peeling agents, polishes, condom(s) (male & female), intrauterine devices (IUDs), diaphragm(s), as additives to existing bioprotective barrier(s), examples of which might include surgical gloves, garment(s) or suit(s), wound protection(s) or prep(s) as either a lyophilized powder or hydrolizable gel, creating a stable complex for activation when in contact with a biologic environment or mammalian membrane, surgical wound site protection, inter-abdominal device(s) or membrane(s), aural, pre or post-operative, a blood-filtering or phores if machine pre or post-contact with blood, respirators, implantable pumps, gauzes, eluding plastics, tablets or films, anti-rejection devices, implantable or other devices, ultrasonic and pneumatic devices.

The formulations described herein may be administered in any suitable manner, including, but not limited to in an oral, sublingual, buccal, nasal, vaginal, rectal, and/or dermal manner. The formulations may be administered through the skin, a body cavity or orifice, or any mucosal membrane. Preferably, formulations are provided through a mucosal membrane.

In general, the layers may be designed in order to provide the final formulation with a height, width or length of about 0.01 inches to about 1 inches (about 0.03-about 2.5 cm). In another example, the layer may be designed in order to provide the final formulation with a height, width or length of about 0.025 inches to about 0.5 inches (about 0.08-about 1.3 cm). The final formulation may be a single height, width or length between about 0.01 inches to about 1.0 inches (about 0.03-about 2.5 cm) or have a varied height, width or length due to contouring between about 0.01 inches to about 1 inches (about 0.03-about 2.5 cm). It is preferable that the formulation be designed for the intended subject. Accordingly, formulations may be adjusted for human or veterinary applications.

The formulations of the delivery system described herein may be manufactured using any suitable methodology. For example, the formulations may be made via wet or dry tableting procedures. The systems described herein may be formulated and administered by methods well known in the art (see Remington: The Science and Practice of Pharmacy).

Advantageously, the formulations described herein can be designed such that they provide immediate release of the treating agent. “Immediate release” refers to the release of the active agent at the time of administration. Thus, the release of the active agent may occur at the moment of contact with the patient. Alternatively, the formulations described herein can be designed to delay the release of the active agent as would be understood by one skilled in the art. Such sustained and/or timed release formulations may be made by sustained release means of delivery devices that are well known to those of ordinary skill in the art, such as those described in U.S. Pat. Nos. 3,845,770; 3,916,899; 3,536,809; 3,598,123; 4,008,719; 4,710,384; 5,674,533; 5,059,595; 5,591,767; 5,120,548; 5,073,543; 5,639,476; 5,354,556, 5,733,566, and 7,714,170 the disclosures of which are each incorporated herein by reference. These pharmaceutical compositions can be used to provide slow or sustained release of one or more of the active compounds using, for example, hydroxypropylmethyl cellulose, other polymer matrices, gels, permeable membranes, osmotic systems, multilayer coatings, microparticles, liposomes, microspheres, or the like. Suitable sustained release formulations known to those skilled in the art, including those described herein, may be readily selected for use with the pharmaceutical compositions of the invention. Thus, the release of the active agent may occur after a period of time from the moment of contact with the patient. Additionally formulations may include a combination of delayed release and immediate release.

In some formulations, the entire formulation will dissolve and not require removal by the patient. In alternate embodiments, the formulation will not totally dissolve. In some embodiments, about 2 to about 100% of the formulation may dissolve. In preferred embodiments about 2, 5, 10, 15, 20, 25, 30, 35, 40, 45, 50, 55, 60, 65, 70, 75, 80, 85, 90, 95, or 100% of the formulation may dissolve. Embodiments may include an insoluble center to the formulation. An insoluble center can aid a patient to determine if the active agents were properly dispensed, and prevent chewing of the solids. It is preferable if the insoluble center is comprised of substantially comprised of an inert, non-biologically active material.

Advantageously, the formulations described herein can be shaped to aid proper and/or effective administration. As mentioned above, patients often fail to properly administer certain non-invasive prior art formulations. For example, a patient may not understand that sublingual absorption does not involve chewing and swallowing a formulation, and the subject would proceed to simply chew and swallow a sublingual formulation instead of allowing the formulation to dissolve and absorb under the tongue.

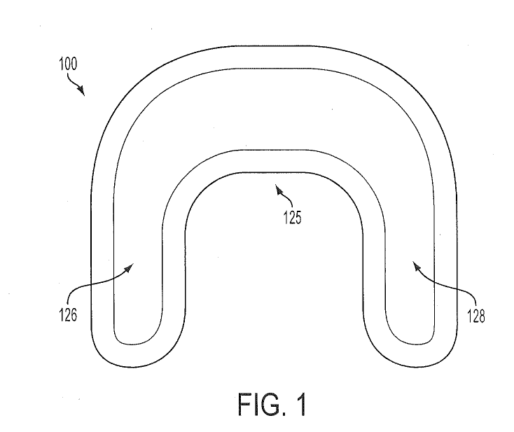

As shown in FIGS. 1-6, the formulations described herein may be shaped into a visually recognizable shape or form, such as a horseshoe, boomerang, or other form fitting or even suggesting behind the teeth of the subject. Preferably, the design of the formulation allows the solid or gel matrix to be easily inserted into the mouth and also allows the patient to recognize when the formulation is properly located with in the mouth. Preferred forms can prevent the formulation from easily dislodging from its intended absorption area, thereby allowing the delivery system to provide the intended therapeutic benefit to the patient.

For example, FIG. 1 illustrates an example of a boomerang-shaped formulation 100. This shape is made to be inserted under the tongue and the shape itself suggests where the formulation of the pharmaceutical should be placed. The size of the formulation can be adjusted to allow insertion into the smallest or largest mouth. In some embodiments, the delivery system maybe customized for individual patients.

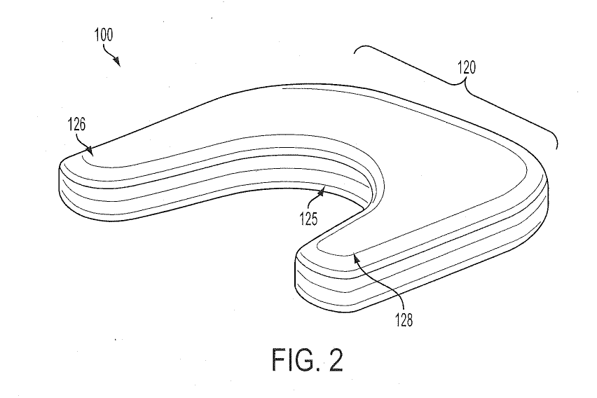

FIG. 2 further illustrates the exemplary shape of formulation 100 from FIG. 1. The general boomerang shape inherently directs the patient towards proper insertion. Formulation 100 may have an underside concave surface portion 130 and a top convex surface portion 120, which aid in maintaining the tablet “in position” under the tongue. The concave surface portion 130 below the tongue prevents rubbing of the delicate tissue (e.g., the frenulum linguae). Cutout portion 125 provides formulation 100 with the distinct boomerang shape and is specifically designed in order to provide adequate wing portions 126, 127 (e.g., length, width and/or height) to prevent formulation 100 from dislodgement due to tongue movement. Preferably, the shape of formulation 100 would prevent or reduce the tendency to chew or swallow formulation 100.

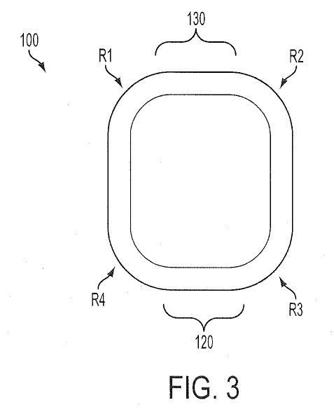

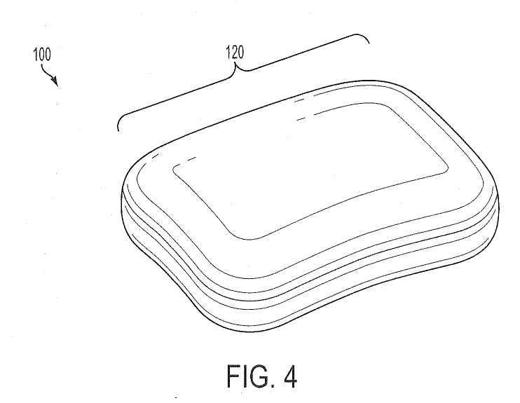

FIG. 3 and FIG. 4 illustrate examples of an oblong, substantially round, or round formulation 100. Preferably this formulation is shaped and sized to easily fit under the tongue. The shape of formulation 100 instructs the patient towards sublingual non-invasive delivery administration, rather than suggesting the ingestion of the formulation.

FIG. 4 further illustrates the shape of formulation 100 from FIG. 3. In this example, formulation 100 can be an oblong tablet that comprises a concave bottom surface (not shown) and convex top surface 120. The shape of formulation 100 preferably provides the patient with positive identification or understanding that formulation 100 is a sublingual tablet that should not be swallowed.

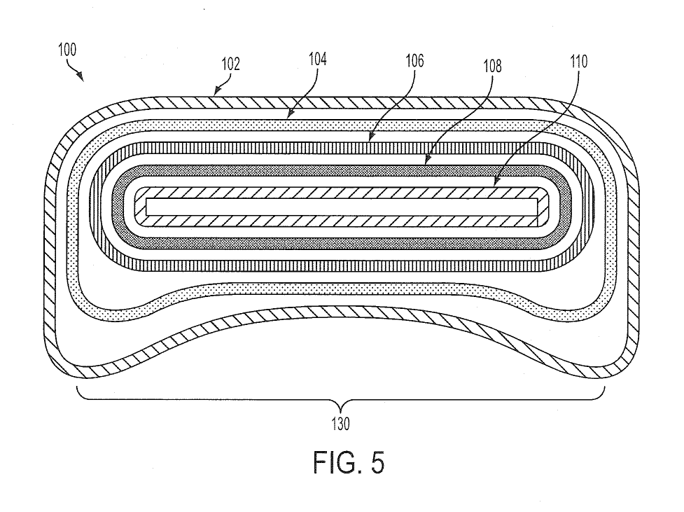

FIG. 5 illustrates an example of a cross-sectional view of the formulation 100 of FIG. 1. Formulation 100 can have concave surface 130. Concave surface 130 provides relief to the mouth under the tongue to prevent rubbing on the tongue (e.g., the frenulum linguae). In embodiments, a flat, convex or concave top surface of formulation 100 may be used. In the present example, formulation 100 has convex surface 120, which provides support to the formulation.

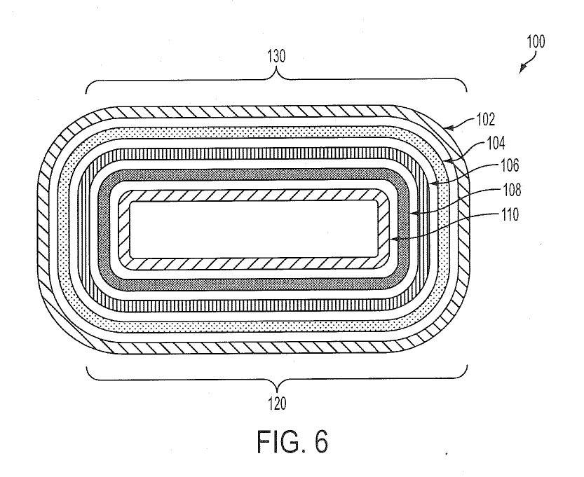

FIG. 6 illustrates an example of a cross-sectional view of the formulation 100 of FIG. 3. In the present example, formulation 100 can have flat top surface 120 and flat bottom surface 130.

FIGS. 5 and 6 also illustrate examples of formulations having various layers. Preferably, the layers are structured to allow the formulation to achieve its intended affect. For example, acidic layer 102 provides an initial acidic layer to condition the mouth to accept the active ingredient and provide a stable environment for the dissolution of the active ingredients in their solid form. Preferably, acidic layer 102 may correspond with, or include, Ingredient F. Additionally, active ingredient layer 104 may preferably include the active ingredient (e.g., Ingredient E) and may also include other adjuvants (e.g., Ingredients A and B). Examples of other adjuvants include vitamin B12 and/or other additives (e.g., arginine). It is preferable that the slow dissolve buffer layer 106 controls the absorption of the outside layers of compounds of formulation 100. Dissolution of slow dissolve buffer layer 106 may control the absorption though modulation of the pores and the ionic strength of the liquid interface in the mouth (or other absorption sites) to the membranes. This layer may be composed of amino acids, salts, and buffering agents. Sugar and sugar alcohol layer 108 may comprise, for example, one or more of sorbitol, erythritol, mannitol, lactose, xylitol, xylose, fructose, glucose, and/or other various sugars that may control and enhance absorption of the active components. Sugar and sugar alcohol layer 108 may correspond to Ingredient C. Preferably, the pH changing layer 110 provides the pH “sweep” and may comprise Ingredient F from above. In the case of an acidic to basic pH sweep, pH changing layer 110 may be comprised of sodium bicarbonate or potassium (bi)carbonate or other basic compounds that would have the property of neutralizing the acid from acidic layer 102. In some embodiments, the dissolution of formulation 100 would provide a mildly basic environment in the mouth (or other absorption site).

The formulation 100 described in FIGS. 5 and 6 are for exemplary and are not to be considered limiting. Preferably, the layers of a formulation may be chosen such that any layer may be interchanged, exchanged, reordered, and repeated with any other layer. The layers may be in any order. Formulations may include any number of layers, either greater, lesser or equal to those illustrates in FIGS. 5 and 6. It is contemplated that the layers may be eliminated or repeated in a variety of different fashions in order to obtain the enhanced effects of absorption and/or pH and ionic strength control. Thus, the layers of a formulation according to the system described herein may be ordered in a different fashion from what is shown in FIGS. 5 and 6.

As stated above, the layers of the formulation may be designed in any order. However, the order of the layers may be relevant to the function of the formulation. For example, if the formulation comprises a basic-sensitive active ingredient, it is advantageous to perform the pH sweep such that the acidic layer is towards the outside surface of the formulation, and the basic ingredients are located towards of the center of the formulation. Such a design is exemplified by of the formulations disclosed in FIGS. 5 and 6.

If, for example, the formulation comprises an acid-sensitive active ingredient, it is advantageous to perform the pH sweep such that the basic layer is towards the outside surface of the formulation, and the acid ingredients are located towards of the center of the formulation. Such a design would be opposite of the formulations disclosed in FIGS. 5 and 6.

The layers of the formulation can be solid, semi-solid, gel, or liquid-state layers. The formulation can comprise layers of a single state (e.g., all layers are solid layers, or all layers are gel layers). The physical state may relate to the function of the formulation. For example, it may be desirable for the formulation to comprise gel layers. Gel layers may expedite the dissolution of the formulation and thereby facilitate the pH sweep. As would be understood by one skilled in the art, this can be useful when the active ingredient is not stable over the entire pH range of the dissolution. In addition, a gel layer may provide a timely rapid change of pH that would halt or decrease the absorption of the active ingredient, thereby helping to control the contact time of the tablet in the absorption area. A gel formulation may also enhance stickiness or bioadhesion, thereby preventing the formulation from being dislodged, and may have a more acceptable tactile and comfort feel to the patient.

Formulations of the delivery system comprise of a variety of different ingredients that perform the functions noted above. Since there are a variety of different methods of administration of any given active agent, there are variations that can be made to accommodate the different types of administration. For example, certain proteins are very sensitive to basic cleavage. In these variations, it is contemplated using an acidic to basic pH sweep of the dissolving formulation. Thus the outer layers of the formulation may comprise the acidic component and the “active protein.” The inner layers, in turn, may comprise more basic components that neutralize the acidic compounds and turn the environment of the protein more basic as the formulation dissolves.

In a particular embodiment, the basic component is coated with a slow-dissolving coating (e.g., zein) and thoroughly mixed with the acidic components. This allows all the ingredients to be formulated as a single tablet.

In another embodiment, certain active agents (e.g., proteins) are difficult to administer as a solid but can be administered as a separate liquid solution. In such an embodiment, the basic ingredient is coated with a slow dissolution coating and is then mixed with the active agent solution, acidic component, and the coated basic component as a liquid immediately prior to administration. The liquid can be administered until the basic components have fully dissolved and the pH has been “swept.”

In yet another embodiment, the formulation may comprise a buffered pH material when the desired and optimal pH of the mixture is known. The buffered pH material can be phosphate salts and organic acid salts, for example, and serve to provide the correct pH and buffer capacity to the administered form of the formulation. The buffered pH material may be an ionized element with a protein ligand, which in certain variations would involve the use of Vitamin B12 and/or chlorophyll ligands.

In other embodiments, a programmed pH change can be designed by encapsulating either the acidic component or basic component of the pH adjuster vehicle. If the acidic components are encapsulated or otherwise slowed in their dissolution while the basic components are allowed to quickly dissolve, a “programmed” pH change from basic to acidic is achieved. Conversely, if the basic component is encapsulated or otherwise slowed in their dissolution while the acidic components are allowed to quickly dissolve a “programmed” pH change from acidic to basic can be formed.

Furthermore, various additional additives, adjuvants or pharmaceutically acceptable excipients (collectively referred to herein as “excipients”) may be incorporated into the embodiments. Optional excipients include, without limitation, binders, excipients, stabilizers, adhesives, lubricants, plasticizers, disintegrants, colorants, bulking substances, flavorings, sweeteners, pH modifiers, buffers, adsorbents, starches, sugars, fats, antioxidants, amino acids, proteins, carotenoids, and derivatives thereof or combinations thereof. More specific excipients include, but are not limited to, pharmaceutical grades of mannitol, lactose, starch, magnesium stearate, sodium saccharin, talcum, cellulose, glucose, sucrose, magnesium carbonate, and the like. One skilled in the pharmaceutical arts would understand the use and selection of excipients, which preferably do not have a therapeutic affect.

The high efficiency of the delivery system described herein allows for decreased dosing of therapeutic active agents and decreased levels of associated toxicities. Additionally the increased stability of the formulations allows the use of biologically and chemically unstable molecules (e.g., proteins) that may have decreased shelf lifetimes. The manufacture and formulations described herein, with visual recognition cues, and layered formulations, combine to solve the problems of non-invasive delivery systems currently known.

### EXAMPLES

### Example 1.0

A sildenafil citrate formulation allows for a significantly increased onset-of-action, providing a more rapid response than the commercially available formulation of sildenafil citrate. The sildenafil citrate/bremelanotide combination formulation of the present invention has been shown to provide similar efficacy, as was reported in clinical trials of conventional bremelanotide or sildenafil citrate but with less toxicity and a more rapid onset-of-action.

### Example 1.1

The exemplary formulation includes 1.00 g powdered sildenafil citrate (120 mesh), 0.300 g of citric acid, 0.20 g of potassium bitartarate, 150 mg of bremelanotide (120 mesh), and 50 mg of vitamin B12 (120 mesh or finer), 1.00 g of sucrose (120 mesh), and 50 mg of stearic acid is added to a 100 mL plastic beaker. After the dry powders have been thoroughly mixed, the powder mixture may be lightly pressed into 50 tablets that are sufficiently pressed to be handled without breakage as known by persons skilled in the manufacture of tablets. To these tablets are added a thin layer of sorbitol (120 mesh) sufficient to cover the surface of the tablet half Each tablet is then pressed with an equally proportional amount of a mixture of 1.5 g of fructose (120 mesh or finer), 750 mg of sodium bicarbonate (120 mesh), and 25 mg of magnesium stearate to give a “double layer” of the tablet. This basic portion of the tablet is then coated with a layer of a protective coating that takes a few seconds more to dissolve than the acidic portion of the tablet. A dip coating of zein (3 g in 100 mL in 95% ethanol) on the basic portion of the tablet is added.

Each tablet is then administered to a patient as a sublingual (under the tongue) dose that is allowed to dissolve in the mouth over a period of 1 second to three minutes with a minimal residence time of 30 seconds under the tongue.

The tablets comprise:
Ingredient A: vitamin B12 (50 mg), (this is protein ligand on a cobalt metal ion);
Ingredient B: none;
Ingredient C: bremelanotide (150 mg) (an active ingredient protein that will interact with vitamin B12);
Ingredient D: potassium bitartarate (0.20 g), (this provides the starting pH point along with the citric acid);
Ingredient E: powdered sildenafil citrate (optional) (1.00 g) and bremelanotide (150 mg); and
Ingredient F: citric acid (0.300 g) and sodium bicarbonate (encased in zein) (750 mg).

The sorbitol is added to separate the acidic components and the basic components. The zein coating is present to allow the acidic side of the tablet to dissolve first, and then the basic side of the tablet is allowed to dissolve and “sweep the pH range.”

### Example 2

An etanercept formulation allows for the non-invasive absorption of a greater than 200 kD molecule across a membrane.

To the dry etanercept powder is added the following: vitamin B12 (methyl coalmine) in a molar amount equivalent to the molar amount of etanercept; vitamin B12 (adenosil colbalamine) in a molar amount equivalent to the molar amount of etanercept; NaHCO₃ coated with sorbitol in the amount of 1 to 10 times the molar amount of etanercept, PDS-01-19 formulation; chlorophylin in the amount of 0.25 molar equivalents of etanercept; L-arginine powder in the amount of 0.5 molar equivalents of etanercept; MSM in the amount of 2.0 molar equivalents of etanercept; sorbitol in the amount of 2.0 molar equivalents of etanercept; xylitol in the amount of 0.5 molar equivalents of etanercept; xylose in the amount of 2.0 molar equivalents of etanercept; and sodium chloride in an amount to make the total ionic strength of the mixture when diluted to approximately the same as the ionic strength of a normal human being. The amount varies depending on the mode of application of the formula,

The dry mixture is then used to form a tablet as described in previous example. Alternatively, the dry mixture can be dispensed as a finely ground powder directly into the mouth for use as a sublingual, or, as described in other sections, as a topical skin absorption formulation or onto a mucous membrane area.

In the case of etanercept, adjustment of the pH to approximately 8.0 led to substantially increased rates of membrane transport. At lower pH values the rate of absorption was dramatically reduced by at least a factor of five times.

Elimination of each of the various cofactors used in the formulation for the transport of etanercept did not help the transport of the drug. In fact, in most tests, slight to substantial decreases in the transport of etanercept were noted.

### Example 3

Certain formulations comprising a variety of active agents were tested in vitro to determine the efficacy of the drug delivery prior to animal or human testing. Generally, in the in vitro experiments a formulation containing the active agent or a control is added to a High Powered Liquid Chromatography 17 mm diameter vial. A flat section of either pig or sheep intestinal membrane is mounted across the open top of the vial. A screw top having a 7 mm hole (approximate) is then screwed to the vial, thereby securing the membrane to the vial. The vial is inverted and immersed in an isotonic solution (e.g., saline). Diffusion of the formulation is then allowed to proceed. The isotonic solution may be agitated with a magnetic stirrer. A pipette is used to sampling the saline solution at designated time intervals (e.g., 5, 10, 30, 60, 90, 180, and 600 seconds).

Other membranes may be used, including commonly available natural casing for food preparation, or cell culture human membranes (e.g. membranes from MatTek Corporation; Ashland, Mass.). Control samples are diluted active agents without any additional adjuvants. Samples may contain vitamin B12 and arginine. The pH of the formulation may be adjusted as necessary. Transport of etanercept occurred at a pH of 8.

### Example 4

The following compounds were analyzed in in vitro tests and the pH of the solutions were adjusted to the values noted below with either sodium bicarbonate (solid) or citric acid to produce the desired testing pH solutions. Using the procedures noted in Example 3, the solution was introduced to the test membranes. The following pH ranges were found to produce rapid diffusion across the membrane. In all cases elimination of the non-invasive delivery formulation produced little if any noticeable diffusion across the membrane in the same time periods tested.

TABLE 1

| | Optimal pH Conditions with |
|---|---|
| Name | Experimental Formulation |
| Atropine sulfate | pH 6.5 to 5.0 |
| Bremelanotide | pH 6.0 to 8.0 |
| Epinephrine | pH 5.0 to 7.0 |
| Neupogen | pH 7.4 |
| Sildenafil | pH 6.8 |
| Enoxaparin Sodium | pH 8.0 to 6.0 |
| Teriparatide (rDNA Origin) | pH 6.8 |
| Adalimumab | pH 7.7 |
| Etanercept | pH 8 |

### Example 5

Formulations comprising a variety of active agents were also tested in vivo. A 300 ml volume/400 g animal formulation was administered sublingually to rats (Sprague Dawley rats, with jugular catheters were purchased from Charles River Laboratories). All rats were in the 275-300 g weight range.

All dose formulations were prepared on the day of dosing. Prior to dose administration, each rat was anesthetized with ketamine:xylazine (7:1, 60 mg/kg, IM) and positioned (supine) so that the head and shoulders were in alignment to preclude drainage of the dose into the trachea or esophagus. The mouth was opened and the interior (including under the tongue) was swabbed with dry cotton applicators. The tongue was held up and the dose was dispensed (same technician for all applications) into the sublingual pocket. The tongue was allowed to return to a normal position and the mouth was allowed to close.

The specific dosages administered to formulations are displayed in Table 1 below. Blood samples were drawn via catheter or cardiac puncture either 10 minutes following administration, or over a time range including 30 seconds, 1 minute, 5 minutes and 15 minutes following administration. The concentration (ng/mL) of the active agent in rat plasma was calculated using high powered liquid chromatography (HPLC). Efficiency was calculated as (ng/mL administered active agent in serum)/(ng/mL active agent in serum from published studies).

TABLE 2

| | | | Observed |
|---|---|---|---|
| | Control Dose | Experimental Dose | Plasma |
| Active Agent | (conventional) | Of Formulation | Levels |
| Bremelanotide | 0.143 mg/kg | 0.194 mg/kg | 136.00% |
| sildenafil citrate | 1.429 mg/kg | 2.42 mg/kg | 169.00% |
| Epinephrine | 0.0043 mg | 0.00172 mg to .0051 mg | 0 to 600% |

The in vivo results show that administered formulations contained significantly smaller dosages, but resulted in much higher blood level serum concentrations per dose that the conventional formulations. Thus, it takes less active agent when administered via the experimental formulation to achieve the same therapeutic benefit as seen in traditional modes of administration. One result would be a significant cost savings for the manufacturer and the patient, resulting in increase commercial availability, including to economically-limited patient populations.

### Example 6

An epinephrine formulation would allow for the non-invasive absorption of a epinephrine across a membrane for the treatment or prevention of anaphylactic shock.

An exemplary formulation would include 1.00 g powdered sildenafil citrate (120 mesh), 0.300 g of citric acid, 0.20 g of potassium bitartarate, 150 mg of bremelanotide (120 mesh), and 50 mg of vitamin B12 (120 mesh or finer), 1.00 g of sucrose (120 mesh), and 50 mg of stearic acid is added to a 100 mL plastic beaker. After the dry powders have been thoroughly mixed, the powder mixture may be lightly pressed into 50 tablets that are sufficiently pressed to be handled without breakage as known by persons skilled in the manufacture of tablets. To these tablets are added a thin layer of sorbitol (120 mesh) sufficient to cover the surface of the tablet half. Each tablet is then pressed with an equally proportional amount of a mixture of 1.5 g of fructose (120 mesh or finer), 750 mg of sodium bicarbonate (120 mesh), and 25 mg of magnesium stearate to give a “double layer” of the tablet. This basic portion of the tablet is then coated with a layer of a protective coating that takes a few seconds more to dissolve than the acidic portion of the tablet. A dip coating of zein (3 g in 100 mL in 95% ethanol) on the basic portion of the tablet is added.

Each tablet would then be administered to a patient as a sublingual (under the tongue) dose that is allowed to dissolve in the mouth over a period of 1 second to three minutes with a minimal residence time of 30 to 90 seconds under the tongue.

The tablets would comprise:

Ingredient A: vitamin B12 (50 mg);

Ingredient B : sodium chloride (5 mg);

ingredient C: arginine (150 mg);

Ingredient D: potassium bitartarate (0.20 g);

Ingredient E: epinephrine (150 mg); and

Ingredient F: citric acid (0.300 g) and sodium bicarbonate (encased in zein) (750 mg).

The sorbitol would be added to separate the acidic components and the basic components. The zein coating would allow the acidic side of the tablet to dissolve first, and then the basic side of the tablet is allowed to dissolve and “sweep the pH range.”

It is to be understood that the invention is not to be limited to the exact configuration as illustrated and described herein. Accordingly, all expedient modifications readily attainable by one of ordinary skill in the art from the disclosure set forth herein, or by routine experimentation there from, are deemed to be within the spirit and scope of the invention as defined by the appended claims.

### Example 7

The delivery systems of the present invention may be used with many different active ingredients. The use of optional adjuvants may be determined through one or more diffusion studies examining a rate of diffusion across a membrane of the active agent complexed to the ingredients. The rate of diffusion or time necessary to obtain a given concentration (i.e., 10% concentration) is measured by sampling the diffused solution. These measurements may be performed over a range of discrete pH values. As a result, the desired pH for the absorption of the active agent may be determined.

In certain methodologies, amounts and concentrations of ingredients may be added or subtracted from the formulation and tested using diffusion studies, for example, to produce an optimally targeted formula for the particular active agent(s). The amounts and dilutions of the ingredients are calculated and adjusted to produce formulations that may have ionic strengths that are similar to that of normal blood (isotonic).

In some embodiments, a mixture of vitamin B12, arginine, xylose, and mannitol in combination with an active agent has shown to be particularly effective.

While illustrative aspects in accordance with the present invention are disclosed herein, it will be appreciated that numerous modifications and other embodiments can be devised by those of ordinary skill in the art. The aspects described herein can be combined, separated, interchanged, and/or rearranged to generate other embodiments. Therefore, it will be understood that the appended claims are intended to cover all such modifications and embodiments that come within the spirit and scope of the present invention. Many variations and modifications will be apparent to those of ordinary skill in the art.

What is claimed is:

1. A non-invasive delivery system for delivering an active agent by absorption across an epithelial membrane comprising:
(a) an effective amount of an active agent; and
(b) at least one vehicle for facilitating absorption of the active ingredient across the epithelial membrane, said vehicle being selected from the following:
(i) metal-complexed vehicles that reversibly complex with the active agent through ionic interactions; and
(ii) pH-adjusting vehicles that adjust the pH of the non-invasive delivery system from a first pH to a second pH.

2. The non-invasive delivery system of claim 1, wherein the system includes at least one metal-complexed vehicle that includes at least one of the following: an ionizable transition metal, an ionized transition metal, an ionizable metal-containing compound, or an ionized metal-containing compound.

3. The non-invasive delivery system of claim 1, wherein the system includes at least a salt that is ionizable or ionized.

4. The non-invasive delivery system of claim 1, wherein the system includes at least a amino acid, protein, sugar, a detergent, or a combination thereof.

5. The non-invasive delivery system of claim 1, wherein the system includes at least a pH buffer.

6. The non-invasive delivery system of claim 1, wherein the system includes at least one pH-adjusting vehicle, wherein the at least one-pH-adjusting vehicle includes an acidic component and a basic component, present in relative amounts for adjusting the pH of the system.

7. The non-invasive delivery system of claim 6, wherein at least one acidic component is citric acid, acetic acid, tartaric acid, ascorbic acid, benzoic acid, erythorbic acid, fumaric acid, gluconic acid inosinic acid, lactic acid, malic acid, oxalic acid, pectic acid, phosphoric acid, sorbic acid, propionic acid, potassium bitartarate, potassium bitartarate, sodium citrate, citric acid, a phosphoric acid salt, a borax salt, 3-{[tris(hydroxymethyl)methyl]amino}propanesulfonic acid, N,N-bis(2-hydroxyethyl)glycine, tris(hydroxymethyl)methylamine, N-tris(hydroxymethyl)methylglycine, dimethylarsinic acid, an organosulfonic acid derivative, N,N-bis(2-hydroxyethyl)glycin, an amino acid, or any combination thereof.

8. The non-invasive delivery system of claim 6, wherein at least one basic component is sodium bicarbonate, potassium bicarbonate, sodium carbonate, potassium carbonate, cream of tartar, an organic amine, a metal salt carbonate, chlorophyll, demetallized chlorophyll, dipotassium tartarate, an organic amine, a pyridine, a pyrimidine, a pyradazine, a quinazoline, a quinoxaline, a quinazoline, a purine, and an nitrogen containing organic base, or any combination thereof.

9. The non-invasive delivery system of claim 2, wherein the at least one metal-complexed vehicle includes a metallic component that includes at least one of the following: Scandium, Titanium, Vanadium, Chromium, Manganese, Iron, Cobalt, Nickel, Copper, Zinc, Gallium, Germanium, Yttrium, Zirconium, Niobium, Molybdenum, Technetium, Ruthenium, Rhodium, Palladium, Silver, Cadmium, Indium, Tin, Antimony, Lanthanum, Hafnium, Tantalum, Tungsten, Rhenium, Iridium, Platinum, Gold, Mercury, Thallium, Lead, Bismuth, Cerium, Praseodymium, Neodymium, Promethium, Samarium, Europium, Gadolinium, Terbium, Dysprosium, Holmium, Erbium, Thulium, Ytterbium, or Lutetium.

10. The non-invasive delivery system of claim 9, wherein the metal is Cobalt.

11. The non-invasive delivery system of claim 1, wherein the system is in the form of a tablet, a patch, or lotion.

12. The non-invasive delivery system of claim 1, wherein the system can be administered sublingually, topically to the skin, vaginally, or rectally.

13. The non-invasive delivery system of claim 1, having a boomerang shaped multilayered formulation, wherein a first layer is a metal-complexing layer having a metallic component, and wherein a second layer is a pH-adjusting layer having a first sublayer and a second sublayer, wherein the first sublayer is an acidic component, and the second sublayer is a basic component.

14. The non-invasive delivery system of claim 1, wherein the system comprises a formulation having both a metal-complexed vehicle and a pH-adjusting vehicle.

15. The non-invasive delivery system of claim 14, further comprising an amino acid, wherein the amino acid is arginine.

16. A method of treating a disease or condition in a mammal comprising administering a non-invasive delivery system that comprises an effective amount of an active agent and at least one metal-complexed vehicle or at least one pH-adjusting vehicle, wherein the at least one metal-complexed vehicle or the at least one pH-adjusting vehicle reversibly complexes with the active agent through ionic interactions so as to facilitate absorption of the active agent across the epithelial membrane.

17. The method of claim 13, wherein the at least one metal-complexed vehicle or at least one pH-adjusting vehicle is administered sublingually, through the skin, or through a mucosal membrane.

18. The method of claim 13, wherein the pH-adjusting vehicle alters the pH of the non-invasive delivery system from a first pH value to a second pH value in order to facilitate absorption of the active agent across the epithelial membrane.

19. The method of claim 13, wherein the metal-complexed vehicle includes at least one of the following: an ionizable or ionized transition metal or metal-containing compound.

20. The method of claim 13, wherein the non-invasive delivery system includes at least an ionized salt, or a readily ionizable salt, wherein the ionized salt, or readily ionizable salt provides ionic strength to the non-invasive system similar to the ionic strength of the epithelial membrane in order to facilitate absorption of the active agent across the epithelial membrane.
